(19) United States
(12) Patent Application Publication
(10) Pub. No.: US 2011/0294853 A1
(43) Pub. Date: Dec. 1, 2011

(54) Bis Aromatic Compounds for Use in the Treatment of Inflammation

(76) Inventors: Benjamin Pelcman, Uppsala (SE); Maria Almeida, Uppsala (SE); Martins Katkevics, Riga (LV); Peter Nilsson, Uppsala (SE); Wesley Schaal, Uppsala (SE); Kristofer Olofsson, Knivsta (LV)

(21) Appl. No.: 13/063,405

(22) PCT Filed: Sep. 10, 2009
(86) PCT No.: PCT/GB2009/002170

§ 371 Date: Aug. 19, 2011

Related U.S. Application Data
(60) Provisional application No. 61/136,539, filed on Sep. 12, 2008.

(51) Int. Cl. A61K 31/444; A61K 31/195; A61P 29/00; C07D 401/12; A61P 11/00; C07C 233/81; C07C 311/21

(52) U.S. Cl. 514/335; 562/442; 514/563; 562/432; 514/562; 546/261

(57) ABSTRACT

There is provided compounds of formula (I), wherein ring A, D₁, D₂ₐ, D_{2b}, D₃, Y, Y¹, L³ and Y³ have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

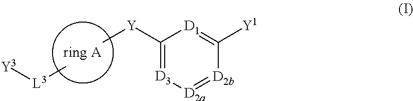

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRAs) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRAs, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are, based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRAs have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomotology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

International patent application WO 2007/113337 discloses a fluorescence based test system, which is employed to measure the formation of the HIV gp41 six-helix bundle. Various biaryl compounds in which a carboxylic acid group is meta relative to the linking point of the biaryl core were the subject of such a test. Further international patent application WO 03/075907 discloses various biaryl compounds that may be useful in inhibiting the entry process of the HIV virus into a mammalian host cell. However, there is no mention in either of these documents of biaryl compounds that are linked with anything other than a methylene group or oxygen atom.

International patent application WO 2005/075410 discloses various compounds for use as medicaments. However, this document does not disclose biaryl ring systems, in which each aromatic ring is further substituted (directly or via a linker group) with another aromatic group.

US patent application US 2005/0014169 and international patent application WO 2004/076640 both disclose various biaryl compounds that may act as nuclease inhibitors, with the latter document further stating that the compounds disclosed therein may be useful in the treatment of cancer. However, there is no mention in either document that the compounds disclosed therein may be useful in the treatment of inflammation.

International patent application WO 2006/125593 and European patent application EP 1 113 000 both disclose compounds that may have potential use in the treatment of inflammation. However, the former document predominantly relates to biaryl ring systems that are not further substituted with aromatic groups, and the latter mainly relates to biaryl compounds containing a cycloalkylamino moiety, but not a carboxylic acid group, or isostere thereof.

International patent applications WO 2007/113254, WO 2005/053609, WO 01/066098, WO 2006/104957, WO 2006/055625, WO 2005/042520 and WO 01/023347, WO 2005/075410, WO 2004/099170, US patent applications U.S. Pat. No. 6,251,917, US 2004/0229891, US 2004/0082641, US 2005/0277640, US 2007/0066660 U.S. Pat. No. 5,447,957, U.S. Pat. No. 5,298,652 and journal article Iyakuhin Kenkyu, by Suzuki et al (1984), 15(2), 195-206 all disclose various biaryl compounds. However, none of these documents mention that the compounds disclosed therein may be useful as inhibitors of LTC₄ synthase, and therefore of use in the treatment of inflammation.

US patent application US 2004/0209882 discloses various methods and compositions of triazine compounds, which may be useful in treating pathophysiological conditions. However, there is no specific disclosure in this document of two aromatic groups linked together, in which each of the aromatic groups are further substituted with another aromatic group.

Journal article Bioorganic & Medicinal Chemistry 12 (2006) 2209-2224 by Li et al discloses various chemical inhibitors of human cyclophilin A. International patent application WO 03/004458 discloses various compounds that may be useful as modulators of the PPARα and PPARγ receptors. However, these documents do not mention compounds that may be LTC₄ synthase inhibitors, and therefore compounds that may be useful in the treatment of inflammation.

Finally, international patent applications WO 2008/107661 and WO 2009/030887 and unpublished PCT application PCT/GB2009/000966 all disclose various compounds for use as LTC₄ synthase inhibitors. However, there is no mention in these documents of biaryl compounds that are linked via certain linker groups.

### DISCLOSURE OF THE INVENTION

There is provided a compound of formula I,

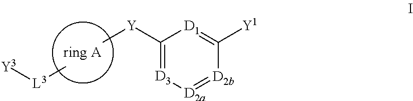

wherein
either one of D₂ₐ and D_{2b} represents D₂, and the other represents —C(-L²-Y²)═;
Y represents a direct bond, —C(R^{b1})(R^{b2})—C(R^{b3})(R^{b4})—, —C(R^{c1})═C(R^{c2})—, —C≡C—, —O—C(R^{d1})(R^{d2})—, —C(R^{e1})(R^{e2})—O—, —N(R^{f1})—C(O)—, —C(O)—N(R^{g1})—, —C(OR^{q1})(R^{h1})—, —N(Rⁱ¹)—C(R^{j1})(R^{j2})—, —C(R^{k1})(R^{k2})—N(R^{m1})—, —N(Rⁿ¹)—S(O)₂— or —S(O)₂—N(R^{p1})—;
R^{b1}, R^{b2}, R^{b3}, R^{b4}, R^{c1}, R^{c2}, R^{d1}, R^{d2}, R^{e1}, R^{e2}, R^{f1}, R^{g1}, R^{h1}, Rⁱ¹, R^{j1}, R^{j2}, R^{k1}, R^{k2}, R^{m1}, Rⁿ¹, R^{p1} and R^{q1} independently represent hydrogen or C₁₋₆ alkyl optionally substituted by one or more substituents selected from ═O, halo and —OR^{s1}; or
Rⁱ¹, R^{m1} and R^{q1} may independently represent —S(O)₂R^{r1}; or
any two of R^{b1}, R^{b2}, R^{b3}, R^{b4}, R^{c1}, R^{c2}, R^{d1}, R^{d2}, R^{e1}, R^{e2}, R^{j1}, R^{j2}, R^{k1} and R^{k2} when attached to the same or adjacent carbons atoms (i.e. the following combinations: R^{b1} and R^{b2}; R^{b3} and R^{b4}; either one of R^{b1} and R^{b2} and either one of R^{b3} and R^{b4}; R^{c1} and R^{c2}; R^{d1} and R^{d2}; R^{e1} and R^{e2}; R^{j1} and R^{j2}; or R^{k1} and R^{k2}) may be linked together to form, together with the carbon atom(s) to which they are attached, a 3- to 8-membered ring optionally containing one to three unsaturations (e.g. triple or, preferably, double bonds), one to three heteroatoms, and which ring is optionally substituted by one or more substituents selected from halo and C₁₋₃ alkyl (optionally substituted by one or more substituents selected from ═O and halo);
R^{r1} represents C₁₋₆ alkyl optionally substituted by one or more fluoro atoms;
R^{s1} represents hydrogen or C₁₋₆ alkyl optionally substituted by one or more fluoro atoms;
each of D₁, D₂ and D₃ respectively represent —C(R^{1a})═, —C(R^{1b})═ and —C(R^{1c})═;
ring A represents:

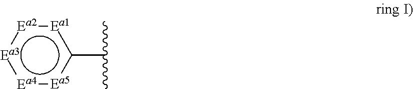

each of E^{a1}, E^{a2}, E^{a3}, E^{a4} and E^{a5} respectively represent —C(R^{2a})═, —C(R^{2b})═, —C(R^{2e})═, —C(R^{2d})═ and —C(R^{2a})═, or, each of E^{a1}, E^{a2}, E^{a3}, E^{a4} and E^{a5} may alternatively and independently represent —N═;
R^{2a} and R^{2e} independently represent hydrogen, —Y^{1a} or a substituent selected from X¹;
one of R^{2b}, R^{2c} and R^{2d} represents the requisite -L³-Y³ group, and the others independently represent hydrogen, —Y^{1a} or a substituent selected from X¹;

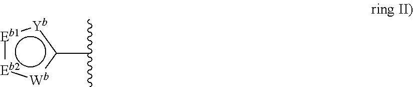

E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, —Y^{1a} or a substituent selected from X², and the remaining R^{3d} substituent (if present) represents hydrogen or a substituent selected from R^{z1}; or

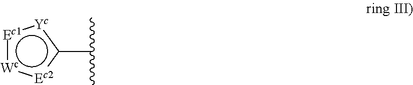

E^{c1} and E^{c2} each respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, —Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b} and R^{1c} independently represent hydrogen, a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent, on each occasion when used herein, —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and —S(O)₂-M¹;
R^{8C}, R^{8f} and R^{8h} independently represent hydrogen, —S(O)₂CH₃, —S(O)₂CF₃ or C₁₋₆ alkyl optionally substituted by one or more substituents selected from F, Cl, ═O, —OR^{13a}, —N(R^{14a})R^{14b} and —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O and/or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent hydrogen, —CH₃ or —CH₂CH₃;
Y¹ and Y^{1a} independently represent, on each occasion when used herein, —C(O)OR^{9a};
R^{9a} represents, on each occasion when used herein, hydrogen, C₁₋₈ alkyl, a heterocycloalkyl group (which latter two groups are optionally substituted by one or more substituents selected from G¹ and/or Z¹), an aryl group or a heteroaryl group (which latter two groups are optionally substituted by one or more substituents selected from G¹);
Y² and Y³ independently represent an aryl group or a heteroaryl group, both of which groups are optionally substituted by one or more substituents selected from A;

A represents, on each occasion when used herein:

I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)ᵣA³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)ᵣ— or —S(O)ᵣN(R^{17e})—;
Z¹ represents, on each occasion when used herein, ═O, ═S, ═NOR^{16b}, ═NS(O)₂N(R^{17f})R^{16c}, ═NCN or ═C(H)NO₂;

B represents, on each occasion when used herein:

I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)ᵣA⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)ᵣ or —S(O)ᵣN(R^{19e})—;
Z² represents, on each occasion when used herein, ═O, ═S, ═NOR^{18b}, ═NS(O)₂N(R^{19f})R^{18c}, ═NCN or ═C(H)NO₂;

R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:

i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹-R^{20a};
wherein A¹¹ represents a single bond or a spacer group selected from —C(O)A¹²-, —S—, —S(O)ᵣA¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)ᵣ— or —S(O)ᵣN(R^{21e})—;
Z³ represents, on each occasion when used herein, ═O, ═S, ═NOR^{20b}, ═NS(O)₂N(R^{21f})R^{20c}, ═NCN or ═C(H)NO₂;
each r independently represents, on each occasion when used herein, 1 or 2;

R^{20a}, R^{20b}, R^{20c}, R^{21a}, R^{21b}, R^{21c}, R^{21d}, R^{21e} and R^{21f} are independently selected from:

i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L² and L³ independently represent a spacer group selected from —N(R^{24a})-A¹⁶-, and —OA¹⁷-;
A¹⁶ represents a direct bond, —C(O)—, —C(O)N(R^{25a})—, —C(O)CH₂— or —S(O)₂—;
A¹⁷ represents a direct bond or —CH₂—;
m represents, on each occasion when used herein, 0, 1 or 2;
R^{22a}, R^{22b}, R^{22c}, R^{22d}, R^{22e}, R^{22f}, R^{23a}, R^{23b}, R^{23c}, R^{24a} and R^{25a} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt thereof,
provided that when:
(A) D₂ₐ represents D₂ and D_{2b} represents —C(-L²-Y²)═; ring A represents ring I); E^{a1}, E^{a2}, E^{a3}, E^{a4} and E^{a5} respectively represent —C(R^{2a})═, —C(R^{2b})═, —C(R^{2b})═, —C(R^{2d})═ and —C(R^{2e})═; R^{1a}, R^{1b}, R^{1c}, R^{2a}, R^{2d} and R^{2e} all represent hydrogen; Y¹ and Y^{1a} both represent —C(O)OR^{9a}; R^{9a} represents hydrogen:
(I) Y represents a direct bond; R^{2b} represents —Y^{1a}; R^{2c} represents -L³-Y³:
   (a) L² and L³ both represent —N(H)C(O)—, then Y² and Y³ do not both represent 4-fluorophenyl;
   (b) L² and L³ both represent —N(H)C(O)—, then Y² and Y³ do not both represent: unsubstituted phenyl; unsubstituted 1-naphthyl; 2-fluorophenyl; 3-fluorophenyl; 2-chlorophenyl; 4-chlorophenyl; 3-bromophenyl; 4-bromophenyl; 2-iodophenyl; 4-iodophenyl; 2-aminophenyl; 2-nitrophenyl; 3-nitrophenyl; 2-methylphenyl; 4-tert-butylphenyl; 2-acetoxyphenyl; 4-acetoxyphenyl; 3-acetamidophenyl; 4-acetamidophenyl; 3-(2,2-dimethyl-1-oxopropylamino)phenyl (i.e. 3-(2,2-dimethyl-propionamido)phenyl); 4-(2,2-dimethyl-1-oxopropyl-amino)phenyl (i.e. 4-(2,2-dimethyl-propionamido)phenyl); 4-biphenyl; 4-acetoxyophenyl; 4-methoxyphenyl; 3-ethoxyphenyl; 4-ethoxyphenyl, 4-n-propyloxyphenyl, 3,5-dimethyoxyphenyl; 2-methyl-3-nitrophenyl; 4-methyl-3-nitrophenyl; 4-(4-nitrophenoxy)phenyl; 3,4-dichlorophenyl; 2-chloro-4-nitrophenyl; 2-(carboxy)phenyl; 2-carboxy-4-nitrophenyl; 1,3-dihydro-1,3-dioxo-5-isobenzofuranyl or 4-[(1,3-dihydro-1,3-dioxo-5-isobenzofuranyl)oxy]phenyl;
   (c) L² and L³ represent —N(H)—S(O)₂—, then Y² and Y³ do not both represent 4-methylphenyl;
   (d) L² represents —N(H)—C(O)—; Y² represents 2,4-dichlorophenyl; then L³ does not represent —O— or —O—CH₂— when Y³ represents unsubstituted phenyl;
   (e) L² and L³ represent —N(H)—, then Y² and Y³ do not both represent 5-hydroxy-7-(sulfonic acid)naphth-2-yl, 8-hydroxy-6-(sulfonic acid)naphth-2-yl or 2-anthraquinone;
(II) Y represents a direct bond; R^{2c} represents —Y^{1a}; R^{2b} represents -L³-Y³; L² and L³ both represent —N(H)C(O)—, then Y² and Y³ do not both represent 4-biphenyl;
(B) ring A represents ring I); E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═; R^{9a} represents hydrogen:
(I) Y represents —N(H)—C(O)—; D_{2b} represents D₂; D₂ₐ represents —C(-L²-Y²)═; R^{1a}, R^{1b}, R^{1c}, R^{2a}, R^{2b}, R^{2d} and R^{2e} all represent hydrogen; R^{2c} represents -L³-Y³; L² and L³ represent —N(H)C(O)—, then Y² and Y³ do not both represent 2-carboxy-4-nitrophenyl or 4-amino-2-carboxy-phenyl;
(II) Y represents —CH₂—CH₂—; D₂ₐ represents D₂; D_{2b} represents —C(-L²-Y²)═; R^{1a}, R^{1b}, R^{1c}, R^{2a}, R^{2d} and R^{2e} all represent hydrogen; R^{2b} represents Y^{1a}; R^{2c} represents -L³-Y³; L² and L³ represent —N(H)C(O)—; Y¹ and Y^{1a} both represent —C(O)OR^{9a}, then Y² and Y³ do not both represent 3,5-dimethoxyphenyl;
   (C) Y represents a direct bond; D_{2b} represents D₂ and D₂ₐ represents —C(-L²-Y²)═; ring A represents ring I); E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═; R^{1c} represents chloro; R^{1a}, R^{1b}, R^{2a}, R^{2d} and R^{2e} all represent hydrogen; R^{2c} represents —Y^{1a}; R^{2b} represents -L³-Y³; Y¹ and Y^{1a} both represent —C(O)OR^{9a}; R^{9a} represents hydrogen; L² and L³ both represent —N(H)—, then Y² and Y³ do not both represent 2-carboxyphenyl,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range), defined herein may be straight-chain or, when there is a sufficient number (i.e. a minimum of two or three, as appropriate) of carbon atoms, be branched-chain, and/or cyclic (so forming, in the case of alkyl, a C_{3-q} cycloalkyl group or, in the case of alkylene, a C_{3-q} cycloalkylene group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, in the case of alkyl, a C_{2-q} alkenyl or a C_{2-q} alkynyl group or, in the case of alkylene, a C_{2-q} alkenylene or a C_{2-q} alkynylene group). In the case of alkylene groups, it is preferred that they are acyclic and/or straight-chain, but may be saturated or unsaturated.

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N— or S— oxidised form.

For the avoidance of doubt, the term “bicyclic” (e.g. when employed in the context of heterocycloalkyl groups) refers to groups in which the second ring of a two-ring system is formed between two adjacent atoms of the first ring. Bicyclic also includes bridged bicyclic groups. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic, bicyclic or tricyclic (e.g. monocyclic or bicyclic) and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothiadiazolyl (including 2,1,3-benzothiadiazolyl), benzothienyl, carbazolyl, chromanyl, cinnolinyl, furanyl, imidazolyl, imidazopyridyl (including imidazo[4,5-b]pyridyl, imidazo[5,4-b]pyridyl and imidazo[1,2-a]pyridyl), indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,3,4-thiadiazolyl), thiazolyl, oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-t]pyridyl and, in particular, thiazolo[4,5-c]pyridyl and thiazolo[5,4-c]pyridyl), thiochromanyl, thienyl, triazolyl (including 1,2,3-triazolyl and 1,2,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic (e.g. bicyclic or tricyclic), they are preferably linked to the rest of the molecule via an aromatic ring. Heteroaryl groups may also be in the N— or S— oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which two X¹ groups are present, which both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{5a} and —C(O)R^{5b} in which R^{5b} represents R^{5a}, then the identities of the two R^{5a} groups are not to be regarded as being interdependent. Likewise, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, the first hyphen in the linker groups represented by ‘Y’ is attached to ring A of the compound of formula I, and the second hyphen represents the point of attachment to the D₁ to D₃-containing ring. Hence, in the case of the group —N(R^{f1})C(O)—, the (first hyphen of the) “—N(R^{f1})—” moiety is attached to ring A and the (second hyphen of the) “—C(O)-” moiety is attached to the D₁ to D₃-containing ring.

For the avoidance of doubt, when a term such as “R^{5a} to R^{5h}” is employed herein, this will be understood by the skilled person to mean R^{5a}, R^{5b}, R^{5c}, R^{5d}, R^{5e}, R^{5f}, R^{5g} and R^{5h} inclusively.

For the avoidance of doubt, when the term “an R⁵ group” is referred to herein, we mean any one of R^{5a} to R^{5k}, R^{5m}, R⁵ⁿ or R^{5p}.

For the avoidance of doubt, where it is stated herein that “any pair of R^{16a} to R^{16c} and R^{17a} to R^{17f} . . . may . . . be linked together”, we mean that any one of R^{16a}, R^{16b}, or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—) or R^{16c} and R^{17f} may be linked together with the nitrogen atom to which they are necessarily attached to form a ring as hereinbefore defined.

For the avoidance of doubt, the compounds of the invention relate to either of the following compounds of formula I,

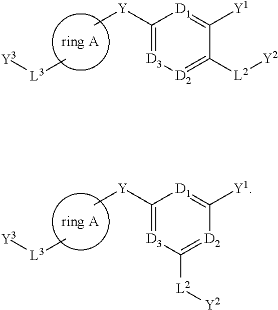

The skilled person will appreciate that, given that there is an essential ‘-L³-Y³’ group present in the compound of formula I, then when, for example, ring A represents ring I), then at least one of —C(R^{2b})═, —C(R^{2c})═ and —C(R^{2d})═ must be present, in which any one of the relevant R^{2b}, R^{2c} and R^{2d} groups represents the essential -L³-Y³ group.

Compounds of the invention that may be mentioned include those in which:

r represents, on each occasion when used herein, 2;
R^{m1} represents hydrogen or C₁₋₆ alkyl optionally substituted by one or more substituents selected from ═O, halo and —OR^{s1} (i.e. R^{m1} may not represent —S(O)₂R^{r1});
R^{s1} represents C₁₋₆ alkyl optionally substituted by one or more fluoro atoms.

Preferred compounds of the invention that may be mentioned include those in which:

when ring A represents ring (II), then one of R^{3a}, R^{3b} and, if present, R^{3c}, represents the requisite -L³-Y³ group, and the remaining R^{3a}, R^{3b} and (if present) R^{3c} substituents represents hydrogen, —Y^{1a} or a substituent selected from X², and the R^{3d} substituent (if present) represents hydrogen or a substituent selected from R^{z1}; or
when ring A represents ring III), then one of R^{4a}, R^{4b} and, if present, R^{4c} represents the requisite -L³-Y³ group, and the remaining R^{4a}, R^{4b} and (if present) R^{4c} substituents represent hydrogen, —Y^{1a} or a substituent selected from X³, and the R^{4d} substituent (if present) represents hydrogen or a substituent selected from R^{z2}.
when R^{3d} and/or R^{4d} (if present) represent a substituent represented by R^{z1} or R^{z2} (i.e. a substituent selected from Z^{1a}), then R^{5c} and R^{5j} independently represent R^{5a} (i.e. R^{5c} and R^{5j} may not represent hydrogen).

Compounds of the invention that may be mentioned include those in which:

Y represents —C(R^{b1})(R^{b2})—C(R^{b3})(R^{b4})—, —C(R^{c1})═C(R^{c2})—, —C≡C—, —O—C(R^{d1})(R^{d2})—, —C(R^{a1})(R^{e2})—O—, —N(R^{f1})—C(O)—, —C(O)—N(R^{g1})—, —C(OR^{q1})(R^{h1})—, —N(Rⁱ¹)—C(R^{j1})(R^{j2})—, —C(R^{k1})(R^{k2})—N(R^{m1})—, —N(Rⁿ¹)—S(O)₂— or —S(O)₂—N(R^{p1})—;
Y represents a direct bond;
Y represents any one of the possible integers defined herein;
when alkyl groups herein are substituted with one or more halo atoms, then those halo atoms are preferably fluoro.

Further compounds of the invention that may be mentioned include those in which, for example when Y represents a direct bond, then A¹⁶ represents —C(O)—, —C(O)N(R^{25a})—, —C(O)CH₂— or —S(O)₂ (especially when L² and/or L³ represent —N(R^{24a})-A¹⁶-).

Compounds of the invention that may be mentioned include those in which for example:

when R^{5a} or R^{8a} to R^{8h} represents optionally substituted C₁₋₆ alkyl, then preferably they are not substituted with both ═O and —OR^{8a}, ═O and —OR^{11a}, or ═O and —OR^{13a} (as appropriate) at the terminal positions of the alkyl group (so forming, for example a —C(O)OR^{8a}, —C(O)OR^{11a} or —C(O)OR^{13a} group);
when R^{5a} or R^{8a} to R^{8h} represents optionally substituted C₁₋₆ alkyl, then preferably they are not substituted with both ═O and —N(R^{8h})R^{8c}, ═O and —N(R^{12a})R^{12b}, or ═O and —N(R^{14a})R^{14b} (as appropriate) at the terminal positions of the alkyl group (so forming, for example a —C(O)N(R^{8h})R^{8c}, —C(O)N(R^{12a})R^{12b} or —C(O)N(R^{14a})R^{14b} group).

Further compounds of the invention that may be mentioned include those in which when (e.g. ring A represents ring (I); and/or R^{1a}, R^{1b}, R^{1c}, R^{a}, R^{2e} and R^{2c} represent hydrogen): Y represents —C(H)═C(H)—; D₂ₐ represents D₂; D_{2b} represents -L²-Y²; R^{2b} represents Y^{1a}; R^{2d} represents -L³-Y³; L² and L³ represent —O—CH₂—, Y¹ and Y^{1a} both represent —C(O)OR^{9a}; R^{9a} represents hydrogen, then Y² and Y³ do not both represent unsubstituted phenyl.

Further compounds of the invention that may be mentioned include those in which

M¹ and M² independently represent —N(R^{15a})R^{15b} or, preferably, —CH₃, —CH₂CH₃ or —CF₃;
R^{11a} and R^{13a} independently represent —CH₂CH₃, —CHF₂ or preferably, hydrogen, —CH₃ or —CF₃.

Compounds of the invention that may be mentioned include those in which, for example, when D₂ₐ represents D₂, and D₁ and D₂ respectively represent —C(R^{1a})═ and —C(R^{1b})═, then:

R^{1a} and/or R^{1b} do not represent —C(O)OR^{5c}, —N(R^{5k})S(O)₂R^{5m}, —C(H)(CF₃)OH, —C(O)CF₃, —C(OH)₂CF₃, —C(CF₃)₂OH or —S(O)₂N(R^{6h})R^{7h} (most particularly R^{1a} and/or R^{1b} do not represent —C(O)OR^{5c});
R^{1a} and R^{1b} independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —OC(O)R⁵ⁿ, —OC(O)OR^{5p} or —OS(O)₂N(R⁶ⁱ)R⁷ⁱ;
for example when R^{1a} and/or R^{1b} represents Z^{2a}, then Z^{2a} preferably represents —R^{5a}, —C(O)N(R^{6a})R^{7a} or —S(O)ₘR^{5j};
for example when Z^{2a} represents —R^{5a}, then R^{5a} preferably represents C₁₋₆ alkyl optionally substituted by one or more substituents selected from ═O or, preferably, halo, —CN, —N₃, —N(R^{8b})R^{8c}, —S(O)ₙR^{8d}, —S(O)₂N(R^{8e})R^{8f} and —OS(O)₂N(R^{8g})R^{8h};
for example when Z^{2a} represents —R^{5a}, then R^{5a} preferably does not represent C₁₋₆ alkyl substituted by more than one substituent, in which the substituents include both: —OR^{8a} and fluoro; and ═O and fluoro;
for example when Z^{2a} represents —R^{5a}, R^{5a} represents C₁₋₆ alkyl substituted by one or more substituents, in which at least one of the substituents is —OR^{8a}, then preferably, R^{8a} represents C₁₋₆ alkyl optionally substituted as hereinbefore defined;
R^{1a} and R^{1b} independently represent —S(O)ₘR^{5j}, or, preferably, hydrogen, —C(O)N(R^{6a})R^{7a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —OC(O)R^{5h}, —OC(O)OR^{5p} or —OS(O)₂N(R⁶ⁱ)R⁷ⁱ.

Further compounds of the invention that may be mentioned include those in which, for example, when ring A represents ring (I) and E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, then:

R^{5a} represents, on each occasion when used herein, C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} and/or —OS(O)₂N(R^{8g})R^{8h};
R^{5a} represents, on each occasion when used herein, C₁₋₆ alkyl optionally substituted by one or more substituents selected from halo, —CN, —N₃, ═O, —N(R^{8b})R^{8c}, —S(O)ₙR^{8d}, —S(O)₂N(R^{8e})R^{8f} and/or —OS(O)₂N(R^{8g})R^{8h};
(e.g. one of) Y² and Y³ represent(s) an aryl group optionally substituted as defined herein.

Further compounds of the invention that may be mentioned include those in which, for example, when ring A represents ring (I); and E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, then:

when R^{1a}, R^{1b}, R^{1c} or, if present, X¹ represent —N(R^{5d})C(O)R^{6c}, and R^{6c} represents R^{5a}, then R^{5a} represents a linear or branched C₁₋₆ alkyl group optionally substituted by one or more substituents selected from halo, —CN, —N₃, ═O, —OR^{8a}, —N(R^{8b})R^{8b}, —S(O)ₙR^{8d}, —S(O)₂N(R^{8e})R^{8f} or —OS(O)₂N(R^{8g})R^{8h};
R^{1a}, R^{1b} and R^{1c} independently represent hydrogen, a group selected from Z^{2a}, halo, —CN, —N(R^{6b})R^{7b}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, halo, —CN, —N(R^{6b})R^{7b}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ.

Yet further compounds of the invention that may be mentioned include those in which:

when, for example, ring A represents ring (I); L² or L³ represent —N(R^{24a})A¹⁶-; A¹⁶ represents a single bond; and/or R^{24a} represents H, then:
Y² or Y³ (as appropriate) do not represent a benzimidazolyl (such as one attached to the L² or L³ group via the imidazolyl moiety, e.g. benzimidazol-2-yl) group;
when Y² or Y³ represents heteroaryl, then it is preferably a monocyclic heteroaryl group or a bicyclic heteroaryl group containing 1 to 4 heteroatoms consisting of 1, 3 or 4 nitrogen heteroatoms, 1 or 2 oxygen heteroatoms and/or 1 sulfur atom, for instance, the bicyclic heteroaryl group may contain 1 nitrogen, oxygen or sulfur heteroatom (all of which are optionally substituted by one or more substituents selected from A);
when Y² or Y³ represents a polycyclic (e.g. bicyclic) heteroaryl group, then it is preferably not attached to the L² or L³ group via a ring containing a heteroatom;
Y² and/or Y³ (as appropriate) represent(s) aryl or a 5- or 6-membered monocyclic ring (all of which are optionally substituted by one or more substituents selected from A).

Further compounds of the invention that may be mentioned include those in which ring A does not represent a triazinyl ring. That is, ring A does not represent ring (I) in which E^{a1}, E^{a3} and E^{a5} all represent —N═.

Further compounds of the invention that may be mentioned include those in which for example when there is an X¹, X², R^{z1}, X³ or R^{z2} substituent present, then:

X¹, X², R^{z1}, X³, R^{z2} do not represent —C(O)N(R^{6a})R^{7a}, in which R^{6a} and R^{7a} represent R^{5a} and R^{5a} represents C₁₋₆ alkyl (e.g. ethyl) terminally substituted with a ═O group (so forming an aldehyde);
for example when R^{6a} and/or R^{7a} represent R^{5a}, then R^{5a} represents, C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} or —OS(O)₂N(R^{8g})R^{8h}.

Preferred compounds of the invention include those in which:

when any two of R^{b1}, R^{b2}, R^{b3}, R^{b4}, R^{c1}, R^{c2}, R^{d1}, R^{d2}, R^{e1}, R^{e2}, R^{j1}, R^{j2}, R^{k1} and R^{k2} are linked together they form, together with the carbon atom(s) to which they are attached, a 5- to 6-membered ring optionally containing one to three double bonds, one to three (e.g. one) heteroatom(s) (e.g. oxygen or, preferably, nitrogen), and which ring is optionally substituted by one or more substituents selected from halo and C₁₋₃ alkyl (optionally substituted by one or more halo atoms), but which ring is preferably unsubstituted;
R^{1a}, R^{1b} and R^{1c} independently represent a group selected from Z^{2a}, —N(R^{5d})C(O)R^{6c}, —N₃, —N(R^{5k})S(O)₂R^{5m}, preferably, halo, —CN, —N(R^{6b})R^{7b}, —NO₂, —OR^{5h}, or, more preferably, hydrogen (most preferably R^{1a}, R^{1b} and R^{1c} independently represent halo (e.g. F and/or Cl) and, especially, hydrogen);
when ring A represents ring (I), then two (e.g. E^{a1} and E^{a2}), preferably, one (e.g. E^{a1} or E^{a2}) or, e.g. more preferably, none of E^{a1}, E^{a2}, E^{a3}, E^{a4} and E^{a5} represent a —N═ group;
E^{a1}, E^{a2}, E^{a3}, E^{a4} and E^{a5} respectively represent —C(R^{2a})═, —C(R^{2b})═, —C(R^{2b})═, —C(R^{2d})═ and —C(R^{2e})═;
only one of R^{2a} to R^{2e}, such as only one of R^{2b}, R^{2c} and R^{2d} (e.g. R^{2b}) may represent —Y^{1a};
R^{2a} and R^{2e} independently represent a substituent selected from X¹ or, more preferably, hydrogen (most preferably R^{2a} and R^{2e} independently represent halo (e.g. F and/or Cl) and, especially, hydrogen);
when one of R^{2a} to R^{2e} (e.g. R^{2b}, R^{2c} and R^{2d}) represents —Y^{1a}, then Y^{1a} is preferably —COOR^{9a}, in which R^{9a} is preferably C₁ alkyl or H;
R^{3c} and R^{3d} independently represent F, Cl, —CH₃, —CF₃ or, more preferably, hydrogen;
for example when ring A represents ring (II) then, one of R^{3a} and R^{3b} represents a substituent X² or, more preferably, H or —Y^{1a}, and the other represents the requisite -L³-Y³ group;
R^{4b} and R^{4c} independently represent F, Cl, —CH₃, —CF₃ or, more preferably, hydrogen;
for example when ring A represents ring (III) then, one of R^{4a} and, if present, R^{4d} represents a substituent X³ or, more preferably, H or —Y^{1a}, and the other represents the requisite -L³-Y³ group;
when any one of R^{3a}, R^{3b}, R^{3c}, R^{3d}, R^{4a}, R^{4b}, R^{4c} or R^{4d} (e.g. R^{3a}, R^{3b}, R^{4a} or R^{4d}) represents —Y^{1a}, then Y^{1a} is preferably —COOR^{9a}, in which R^{9a} is preferably C₁₋₄ alkyl or H;
R^{1a}, R^{1b}, R^{1c} (when such R^{1a}, R^{1b} and R^{1c} groups represent a substituent, i.e. a group other than hydrogen), X¹, X² and X³ independently represent a group selected from Z^{2a}, or, halo, —CN, —N(R^{6b})R^{7b}, —N(R^{5d})C(O)R^{6c}, —N₃, —NO₂, —OR^{5h} or —N(R^{5k})S(O)₂R^{5m} (more preferably such R^{1a}, R^{1b} and R^{1c} groups independently represent hydrogen, or a substituent selected from Z^{2a}, or, halo, —CN, —N(R^{6b})R^{7b}, —N(R^{5d})C(O)R^{6c}, —OR^{5h} or —N(R^{5k})S(O)₂R^{5m}, and each X¹, X² and X³ independently represents a group selected from Z^{2a}, or, halo, —CN, —N(R^{6b})R^{7b}, —N(R^{5d})C(O)R^{6c}, —OR^{5h} or —N(R^{5k})S(O)₂R^{5m});
Z^{1a} and Z^{2a} independently represent —C(O)OR^{5c}, —C(O)N(R^{6a})R^{7a} or, preferably, —R^{5a};
when any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ are linked together, they form a 5- or 6-membered ring optionally substituted by F, —OCH₃ or, preferably, ═O or R^{5a}, and which ring optionally contains an oxygen or nitrogen heteroatom (which nitrogen heteroatom may be optionally substituted, for example with a methyl group, so forming e.g. —N(H)— or —N(CH₃)—);
R^{5c}, R^{5j} and R^{6e} independently represent R^{5a};
when R^{5a}, R^{8a}, R^{8b}, R^{8d}, R^{8e} and R^{8g} represent C₁₋₆ alkyl optionally substituted by one or more halo substituents, then those halo substituents are preferably F or Cl (especially fluoro);
R^{5a} represents C₁₋₆ (e.g. C₁₋₄) alkyl optionally substituted by one or more substituents selected from Cl, —N₃, preferably, ═O, —N(R^{8b}) R^{8c} and, more preferably, F and —OR^{8a};
m and n independently represent 2;
when any one of R^{8a} to R^{8h} (e.g. R^{8a}, R^{8b}, R^{8d}, R^{8e} and R^{8g}) represents C₁₋₆ alkyl substituted by halo, then preferred halo groups are fluoro and chloro (especially fluoro);
R^{8a}, R^{8b}, R^{8d}, R^{8e} and R^{8g} independently represent H or C₁₋₃ alkyl optionally substituted by one or more fluoro atoms;
R^{8c}, R^{8f} and R^{8h} independently represent H, —S(O)₂CH₃, —S(O)₂CF₃ or C₁₋₃ alkyl optionally substituted by one or more fluoro atoms, or the relevant pairs (i.e. R^{8b} and R^{8c}, R^{8e} and R^{8f} or R^{8g} and R^{8h}) are linked together as defined herein;
when R^{8b} and R^{8c}, R^{8e} and R^{8f} or R^{8g} and R^{8h} are linked together, they form a 5- or 6-membered ring, optionally substituted by one or more (e.g. one or two) substituents selected from F, ═O or —CH₃;
M¹ and M² independently represent —N(R^{15a})R^{15b} or, preferably, —CH₃ or —CF₃;
R^{11a}, R^{12a}, R^{12b}, R^{13a}, R^{14a}, R^{14b}, R^{15a} and R^{15b} independently represent —CH₂CH₃, —CF₃ (in the case of R^{11a} and R^{13a}) or, preferably, H or —CH₃;
R^{9a} represents C₁₋₈ alkyl or a heterocycloalkyl group, both of which are optionally substituted by one or more substituents selected from G¹ and/or Z¹;
R^{9a} represents C₁₋₄ (e.g. C₁₋₃) alkyl optionally substituted by one or more halo (e.g. fluoro) atoms or an aryl group (e.g. phenyl) substituted by one or more halo (e.g. fluoro or chloro) atoms;
A represents: aryl (e.g. phenyl) optionally substituted by B; C₁₋₈ alkyl optionally substituted by G¹ and/or Z¹; or G¹;
G¹ represents N₃, —NO₂, or, preferably, halo, cyano or -A¹-R^{16a};
A² represents a single bond or —O—;
A⁴ represents —C(O)N(R^{17d})—, —C(O)O— or, more preferably, a single bond or —C(O)—;
A⁵ represents —C(O)— or, preferably, a single bond;
Z¹ represents ═S, ═NCN, preferably, ═NOR^{16b} or, more preferably, ═O;
B represents: heteroaryl (e.g. oxazolyl, thiazolyl, thienyl or pyridyl) or, more preferably, aryl (e.g. phenyl) optionally substituted by G²; C₁₋₆ alkyl optionally substituted by G² and/or Z²; or, preferably, B represents G²;
G² represents cyano, preferably, —NO₂ or, more preferably, halo or -A⁶-R^{18a} (alternatively, G² represents cyano or, preferably, halo or -A⁶-R^{18a});
A⁶ represents a single bond, —N(R^{19e})A⁹- or —OA¹⁰-;
A⁹ represents —C(O)N(R^{19d})—, —C(O)O— or, more preferably, a single bond or —C(O)—;
A¹⁰ represents a single bond;
Z² represents ═S, ═NCN, preferably, ═NOR^{18b} or, more preferably, ═O;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from hydrogen, aryl (e.g. phenyl) or heteroaryl (which latter two groups are optionally substituted by G³) or C₁₋₈ (e.g. C₁₋₆) alkyl (optionally substituted by G³ and/or Z³), or the relevant pairs are linked together as hereinbefore defined;
when any pair of R^{16e} to R^{16c} and R^{17a} to R^{17f}, or R^{18a} to R^{18c} and R^{19a} to R^{19f} are linked together, they form a 5- or 6-membered ring, optionally substituted by one or more (e.g. one or two) substituents selected from G³ and/or Z³;
G³ represents halo or -A¹¹-R^{20a};
A¹¹ represents a single bond or —OA¹⁵-;
A¹² represents a single bond or, preferably, —N(R^{21b})—;
A¹³ represents a single bond or, preferably, —N(R^{21c})—;
A¹⁴ and A¹⁵ independently represent a single bond, —C(O)— or —S(O)₂—;
Z³ represents ═S, ═NOR^{20b} or, preferably, ═O;
R^{20a}, R^{20b}, R^{20c}, R^{21a}, R^{21b}, R^{21c}, R^{21d}, R^{21e} and R^{21f} are independently selected from H, C₁₋₃ (e.g. C₁₋₂) alkyl (e.g. methyl) optionally substituted by one or more halo (e.g. fluoro) atoms, or optionally substituted aryl (e.g. phenyl), or the relevant pairs are linked together as defined herein;
when any pair of R^{20a} to R^{20c} and R^{21a} to R^{21f} are linked together, they form a 5- or 6-membered ring, optionally substituted by one or more (e.g. one or two) substituents selected from halo (e.g. fluoro) and C₁₋₂ alkyl (e.g. methyl);
R^{22a}, R^{22b}, R^{22c}, R^{22d}, R^{22e}, R^{22f}, R^{23a}, R^{23b}, R^{23c}, R^{24a} and R^{25a} independently represent hydrogen or C₁₋₂ alkyl optionally substituted by ═O or, more preferably, one or more fluoro atoms.

More preferred compounds of the invention include those in which:

when ring A represents ring (I), in which there is one —N═ group present, then E^{a1}, E^{a3} or E^{a5} represents such a group;
when ring A represents ring (II), then W^{b} may represent —N(R^{3d})— (so forming a pyrrolyl or imidazolyl ring) or, more preferably, when Y^{b} represents —C(R^{3c})═, then W^{b} preferably represents —O— or, particularly, —S— (so forming a furanyl or, particularly, a thienyl ring) or when Y^{b} represents —N═, then W^{b} preferably represents —O— or —S— (so forming, for example, an oxazolyl or thiazolyl ring);
R^{3c} and R^{3d} independently represent H;
when ring A represents ring (III), then W^{c} preferably represents —N(R^{4d})—;
R^{4d} represents H;
X¹, X² and X³ independently represent halo (e.g. fluoro or chloro; especially fluoro), —CN, —NO₂, —OR^{5h} or Z^{2a};
R^{5h} represents R^{5a};
Z^{2a} represents —R^{5a};
R^{5a} represents C₁₋₄ alkyl (such as methyl, ethyl and isopropyl) optionally substituted by one or halo (e.g. fluoro), so forming for example a difluoromethyl or trifluoromethyl group;
R^{8a}, R^{8b}, R^{8c}, R^{8d}, R^{8e}, R^{8f}, R^{8g} and R^{8h} independently represent H or C₁₋₃ alkyl optionally substituted by one or more fluoro atoms.

Preferred rings that ring A may represents include imidazolyl (e.g. 2-imidazolyl), preferably, furanyl (e.g. 2-furanyl), thienyl (e.g. 2-thienyl), oxazolyl (e.g. 2-oxazolyl), thiazolyl (e.g. 2-thiazolyl), pyridyl (e.g. 2- or 4-pyridyl), pyrrolyl (e.g. 3-pyrrolyl), imidazolyl (e.g. 4-imidazolyl) or, more preferably, phenyl. Alternatively, other preferred rings that A may represents include furanyl (e.g. 2-furanyl), thienyl (e.g. 2-thienyl), imidazolyl (e.g. 2-imidazolyl), oxazolyl (e.g. 2-oxazolyl), thiazolyl (e.g. 2-thiazolyl), or preferably pyridyl (e.g. 3-pyridyl) or phenyl.

Preferred rings that the D₁ to D₃-containing ring may represent include 2-, 3- or 4-pyridyl or, preferably, phenyl.

Preferred aryl and heteroaryl groups that Y² and Y³ may independently represent include optionally substituted (i.e. by A) phenyl, naphthyl, pyrrolyl, furanyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include pyridyl (e.g. 3-pyridyl), benzofuranyl (e.g. 5-benzofuranyl), isoquinolinyl (which may be partially saturated, for example forming 1,2,3,4-tetrahydroisoquinolinyl, e.g. 1,2,3,4-tetrahydroisoquinolin-7-yl) and, more particularly, phenyl. Alternatively, other preferred aryl and heteroaryl groups that Y² and Y³ may independently represent include optionally substituted thienyl (e.g. 2-thienyl), oxazolyl (e.g. 2-oxazolyl), thiazolyl (e.g. 2-thiazolyl), or more preferably, phenyl.

Preferred optional substituents on Y² and Y³ groups include:

—NO₂; or, more preferably
halo (e.g. fluoro, chloro or bromo);
cyano;
C₁₋₆ alkyl, which alkyl group may be cyclic, part-cyclic, unsaturated or, preferably, linear or branched (e.g. C₁₋₄ alkyl (such as propyl (e.g. n-propyl and isopropyl), ethyl or, preferably, butyl (e.g. t-butyl or n-butyl) or methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²⁶;

### —SR²⁶;

### —C(O)R²⁶;

### —C(O)OR²⁶;

### —N(R²⁶)R²⁷; and

### —S(O)₂R²⁸;

wherein R²⁶ and R²⁷ independently represent, on each occasion when used herein, H, C₁₋₆ alkyl, such as C₁₋₅ (e.g. C₁₋₄) alkyl (e.g. ethyl, n-propyl, cyclopentyl, or, preferably, butyl (e.g. t-butyl or, preferably, n-butyl), cyclopropyl, methyl or isopropyl) optionally substituted by one or more halo (e.g. fluoro) groups (so forming e.g. a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms); and R²⁸ preferably represents aryl or, particularly C₁₋₆ alkyl, for example as defined in respect of R²⁶ and R²⁷.

Particularly preferred compounds of the invention include those in which:

Y represents a direct bond, preferably, —O—C(R^{d1})(R^{d2})—, —C(O)—N(R^{g1})—, —N(Rⁱ¹)—C(R^{j1})(R^{j2})—, —S(O)₂—N(R^{p1})—, more preferably, —C(R^{b1})(R^{b2})—C(R^{b3})(R^{b4})—, —C(R^{e1})(R^{e2})—O—, —C≡C—, —N(R^{f1})—C(O)—, —C(OH)(R^{h1})—, —C(R^{k1})(R^{k2})—N(R^{m1})— or —N(Rⁿ¹)—S(O)₂—;
for instance, Y may represent —C(R^{c1})═C(R^{c2})—, preferably, —C(OR^{q1})(R^{h1})— (e.g. —C(OH)(R^{h1})—), or, more preferably, a direct bond, —C(R^{e1})(R^{e2})—O—, —C(R^{k1})(R^{k2})—N(R^{m1})—, —C(R^{b1})(R^{b2})—C(R^{b3})(R^{b4})—, —C≡C—, —N(R^{f1})—C(O)— or —N(Rⁿ¹)—S(O₂)— (alternatively, Y may represent a direct bond, —O—C(R^{d1})(R^{d2})—, —N(Rⁱ¹)—C(R^{j1})(R^{j2})—, —C(R^{b1})(R^{b2})—C(R^{b3})(R^{b4})—, —C≡C—, —N(R^{f1})—C(O)— or —N(Rⁿ¹)—S(O)₂—);
R^{b1}, R^{b2}, R^{b3}, R^{b4}, R^{c1}, R^{c2}, R^{d1}, R^{d2}, R^{e1}, R^{e2}, R^{f1}, R^{g1}, R^{h1}, Rⁱ¹, R^{j1}, R^{j2}, R^{k1}, R^{k2}, R^{m1}, Rⁿ¹, R^{p1} and R^{q1} independently represent C₁₋₄ (e.g. C₁₋₃) alkyl (e.g. methyl;
which alkyl group is optionally substituted by one or more fluoro atoms) or, more preferably, hydrogen;
any two of R^{b1}, R^{b2}, R^{b3}, R^{b4}, R^{c1}, R^{c2}, R^{d1}, R^{d2}, R^{e1}, R^{e2}, R^{j1}, R^{j2}, R^{k1} and R^{k2} may not be linked together,
D_{2b} or, preferably, D₂ₐ represents D₂, and the other (i.e. preferably D_{2b}) represents —C(-L²-Y²);
D₁, D₂ and D₃ respectively represent —C(R^{1a})═, —C(R^{1b})═ and —C(R^{1c})═;
ring A represents ring I) as hereinbefore defined;
E^{a1} represents —N═ or, preferably, —C(R^{2a})═;
E^{a2} represents —N═ or, preferably, —C(R^{2b})═;
E^{a3} and E^{a4} represent —C(R^{2c})═, and —C(R^{2d})═, respectively;
E^{a5} represents —C(R^{2e})═;
R^{2a} and R^{2e} independently represent hydrogen;
one of R^{2b} or R^{2c} (preferably R^{2c}) represents the requisite -L³-Y³ group and the other represents a substituent selected from X¹ or, preferably, hydrogen or —Y^{1a};
R^{2d} represents hydrogen;
X¹, X² and X³ independently represent —OR^{5h}, Z^{2a}, or, most preferably halo (e.g. chloro or, especially, fluoro) (e.g. X¹ represents fluoro);
R^{9a} represents hydrogen or C₁₋₆ (e.g. C₁₋₄) alkyl (such as butyl, e.g. t-butyl, or methyl);
Y² and Y³ independently represent aryl (e.g. phenyl) or heteroaryl (e.g. a monocyclic 5- or 6-membered or a bicyclic 9- or 10-membered heteroaryl group preferably containing one to three heteroatom(s) selected from sulfur or, particularly, nitrogen or oxygen, so forming for example pyridyl), both of which are optionally substituted by one or more (e.g. one to three) substituents selected from A;
A represents I) C₁₋₈ (e.g. C₁₋₆) alkyl (e.g. n-butyl, t-butyl or methyl) optionally substituted by one or more substituents selected from G¹; or II) G¹;
G¹ represents —NO₂ or, preferably, halo (e.g. fluoro or chloro), cyano or -A¹-R^{16a};
A¹ represents a single bond, —C(O)A²-, —S—, —S(O)₂A³-, —N(R^{17a})A⁴- or —OA⁵-;
A², A³, A⁴ and A⁵ independently represent a single bond;
R^{16a} represents hydrogen or C₁₋₈ alkyl (such as C₁₋₆ alkyl or C₃₋₅ cycloalkyl, e.g. cyclopropyl, cyclopentyl, butyl, isopropyl, ethyl or methyl) optionally substituted by one or more groups selected from G³;
R^{17a} represents hydrogen or, preferably, C₁₋₆ (e.g. C₁₋₃) alkyl (such as methyl);
G³ represents halo (e.g. fluoro);
R^{24a} and R^{25a} independently represent hydrogen.

Particularly preferred compounds of the invention include:

Y represents a direct bond, preferably, —C(O)(H)—, —S(O)₂N(H)—, —OCH₂—, —N(H)—CH₂— or, more preferably, —C(OH)(H)—, —CH₂CH₂—, —CH₂—O—, —N(H)C(O)—, —N(H)S(O)₂— or —CH₂—N(H)—;
D₂ₐ represents D₂;
D_{2b} represents —C(-L²-Y²)═;
R^{1a}, R^{1b} and R^{1c} independently represent hydrogen;
ring A represents ring I);
E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═,
R^{2a}, R^{2d} and R^{2e} independently represent hydrogen;
R^{2b} represents hydrogen or —Y^{1a};
R^{2c} represents -L³-Y³;
R^{9a} represents H or C₁₋₄ (e.g. C₁₋₂) alkyl (e.g. methyl);
A¹⁶ represents a —C(O)—, —C(O)N(R^{25a})—, —C(O)CH₂— or —S(O)₂—;
Y² and Y³ independently represent phenyl or pyridyl (e.g. 3-pyridyl), both of which are optionally substituted by one or more substituents selected from A;
A represents C₁₋₄ alkyl (such as butyl or methyl; which alkyl group is optionally substituted by one or more G¹ groups; e.g. halo, such as fluoro, so forming for example a trifluoromethyl group) or G¹;
G¹ represents halo (e.g. fluoro or chloro) or -A¹-R^{16a};
A¹ represents —N(R^{17a})A⁴- or, preferably, —OA⁵-;
A⁴ represents a single bond;
A⁵ represents —C(O)— or, preferably, a single bond;
R^{16a} represents hydrogen (e.g. when A¹ represents —N(R^{17a})A⁴-) or C₁₋₆ (e.g. C₁₋₄ alkyl (e.g. butyl, such as n-butyl, isopropyl or methyl) optionally substituted by one or more halo G³ (e.g. fluoro) groups (so forming for example a trifluoromethyl group);
R^{17a} represents hydrogen;
G³ represents halo (e.g. fluoro).

Preferred Y² and Y³ groups include, e.g. when they represent aryl groups, unsubstituted naphthyl (e.g. 1-naphthyl) and, preferably, phenyl, 2-chlorophenyl, 3-chlorophenyl, 4-chlorophenyl, 4-n-butylphenyl, 4-tert-butylphenyl, 3-trifluoromethylphenyl, 4-trifluoromethylphenyl, 2-methoxyphenyl, 4-methoxyphenyl, 4-isopropoxyphenyl, 4-n-butoxyphenyl, 4-trifluoromethoxyphenyl and 3-chloro-2-methylphenyl. Preferred Y² and Y³ groups include, e.g. when they represent heteroaryl groups, pyridinyl (e.g. unsubstituted 3-pyridyl).

Preferred substituents on Y² and Y³ groups include amino (e.g. NH₂), —C(O)C₁₋₂ alkyl (e.g. —C(O)CH₃), preferably, C₁₋₆ (e.g. C₁₋₄) alkyl optionally substituted by one of more halo atoms (so forming, e.g. trifluoromethyl), or, more preferably, halo (e.g. chloro, bromo or, preferably, fluoro) or C₁₋₆ (e.g. C₁₋₄) alkoxy (e.g. butoxy such as n-butoxy) optionally substituted by one or more halo atoms (so forming, e.g. trifluoromethoxy).

Preferred specific L² and L³ groups that may be mentioned include —N(H)— or, preferably, —N(H)C(O)—, —N(H)C(O)N(H)—, —N(H)S(O)₂—, —O— and —OCH₂—.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which L² and/or L³ represents —N(R^{24a})A¹⁶- in which R^{24a} represents H, reaction of a compound of formula II,

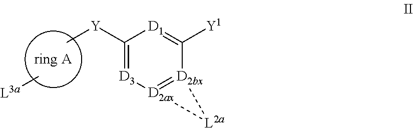

or a protected derivative thereof (e.g. an amino-protected derivative) wherein one of D₂ₐₓ and D_{2bx} represents D₂ and the other represents —C(-L^{2a})═ (i.e. the L^{2a} substituent is attached to either one of D₂ₐₓ and D_{2bx}), L^{2a} represents —NH₂ or -L²-Y², L^{3a} represents —NH₂ or -L³-Y³, provided that at least one of L^{2e} and L^{3a} represents —NH₂, and ring A, D₁, D₂, D₃, Y and Y¹ are as hereinbefore defined, with:
(A) when A¹⁶ represents —C(O)N(R^{25a})—, in which R^{25a} represents H:
(a) a compound of formula III,

Y^{a}—N═C═O; III
or
(b) with CO (or a reagent that is a suitable source of CO (e.g. Mo(CO)₆ or Co₂(CO)₈)) or a reagent such as phosgene or triphosgene in the presence of a compound of formula IV,

Y^{a}—NH₂ IV

wherein, in both cases, Y^{a} represents Y² or Y³ (as appropriate/required) as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). The skilled person will appreciate that for preparation of compounds of formula I in which -L²-Y² represents —C(O)N(H)—Y² and -L³-Y³ represents —C(O)N(H)—Y³ and Y² and Y³ are different, two different compounds of formula III or IV (as appropriate) will need to be employed in successive reaction steps. For the preparation of such compounds starting from compounds of formula II in which both of L^{2a} and L^{3a} represent —NH₂, then mono-protection (at a single amino group) followed by deprotection may be necessary, or the reaction may be performed with less than 2 equivalents of the compound of formula III or IV (as appropriate);
(B) when A¹⁶ represents a direct bond, with a compound of formula VI,

Y^{a}-L^{a} VI

wherein L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y^{a} is as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation;
(C) when A¹⁶ represents —S(O)₂—, —C(O)— or —C(O)CH₂—, with a compound of formula VII,

Y^{a}-A^{16a}-L^{a} VII

wherein A^{16a} represents —S(O)₂—, —C(O)— or —C(O)CH₂—, and Y^{a} and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)-amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);
(ii) for compounds of formula I in which one of L² and L³ represents —N(R^{24a})C(O)N(R^{25a})— (and the other may represent —NH₂ (or a protected derivative thereof, e.g. that contains an aromatic ring), —N(R^{24a})C(O)N(R^{25a})— or another L² or L³ as hereinbefore defined), in which R^{24a} and R^{25a} both represent H, reaction of a compound of formula VIII,

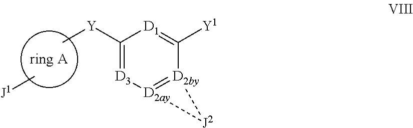

wherein one of D_{2ay} and D_{2by} represents D₂ and the other represents —C(-J²)= (i.e. the J² substituent is attached to either one of D₂ₐₓ and D_{2bx}), one of J¹ or J² represents —N═C═O and the other represents -L²-Y² or -L³-Y³ (as appropriate), —NH₂ (or a protected derivative thereof) or —N═C═O (as appropriate), and ring A, D₁, D₂, D₃, Y and Y¹ are as hereinbefore defined, with a compound of formula IV as hereinbefore defined, under reaction conditions known to those skilled in the art, such as those described hereinbefore in respect of process step (i)(A)(a) above;
(iii) reaction of a compound of formula IX,

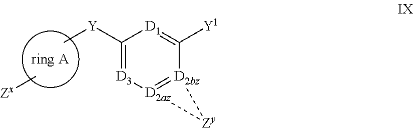

wherein one of D_{2az} and D_{2bz} represents D₂ and the other represents —C(—Z^{y})═ (i.e. the Z^{y} substituent is attached to either one of D_{2az} and D_{2bz}), one of Z^{x} and Z^{y} represents a suitable leaving group and the other represents -L²-Y² or -L³-Y³ (as appropriate) or the other may also represent a suitable leaving group. Suitable leaving groups that Z^{x} and/or Z^{y} may represent include chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃, —BF₃K or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and ring A, D₁, D₂, D₃, Y and Y¹ are as hereinbefore defined, with a (or two separate) compound(s) (as appropriate/required) of formula X,

Y^{a}-L^{x}-H X

wherein L^{x} represents L² or L³ (as appropriate/required), and Y^{a} is as hereinbefore defined, under suitable reaction conditions known to those skilled in the art, for example such as those hereinbefore described in respect of process (i)(B). The skilled person will appreciate that when compounds of formula I in which L² and L³ are different are required, then reaction with different compounds of formula X (for example, first reaction with a compound of formula X in which L^{x} represents —N(R^{24a})A¹⁶-, followed by reaction with another, separate, compound of formula X in which L^{x} represents —OA¹⁷-) may be required;
(iv) compounds of formula I in which there is a R^{f1}, R^{g1}, Rⁱ¹, R^{m1}, Rⁿ¹, R^{p1}, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, or R²⁵ group present, which is attached to a heteroatom such as nitrogen or oxygen, and which does/do not represent hydrogen), may be prepared by reaction of a corresponding compound of formula I in which such a group is present that does represent hydrogen with a compound of formula XI,

R^{wy}-L^{b} XI

wherein R^{wy} represents a R^{f1}, R^{g1}, Rⁱ¹, R^{m1}, Rⁿ¹, R^{p1} or R⁵ to R¹⁹ group in which those groups do not represent hydrogen, and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (i)(C) above. The skilled person will appreciate that various groups (e.g. primary amino groups) may need to be mono-protected and then subsequently deprotected following reaction with the compound of formula XI;
(v) compounds of formula I in which there is a R^{f1}, R^{g1}, Rⁱ¹, R^{m1}, Rⁿ¹, R^{p1} or R⁵ to R¹⁹ group present, which is attached to a heteroatom such as nitrogen or oxygen, and which does/do not represent hydrogen, an aryl group or a hetereoaryl group, may be prepared by reaction of a corresponding compound of formula I in which such a group is present that does represent hydrogen with a compound of formula XII,

R^{wy}-L^{c} XII

wherein R^{wy} represents any R^{wy} group as defined above (as appropriate; and preferably represents an aryl or heteroaryl group (if applicable), for the conversion to the appropriate compound of formula I) as hereinbefore defined, and L^{c} represents a suitable leaving group such as chloro, bromo, iodo, a sulfonate group (e.g. —OS(O)₂CF₃, —OS(O)₂CH₃, —OS(O)₂PhMe or a nonaflate), or a similar group known to the skilled person, under reaction conditions known to those skilled in the art, for example those hereinbefore described in respect of process step (i)(B) above;
(vi) for compounds of formula I that contain saturated alkyl groups, reduction of a corresponding compound of formula I that contains an unsaturation, such as a double or triple bond, in the presence of suitable reducing conditions, for example by catalytic (e.g. employing Pd) hydrogenation;
(vii) for compounds of formula I in which R^{9a} represents hydrogen, hydrolysis of a corresponding compound of formula I in which R^{9a} does not represent H, under standard conditions, for example in the presence of an aqueous solution of base (e.g. aqueous 2M NaOH) optionally in the presence of an (additional) organic solvent (such as dioxane), which reaction mixture may be stirred at room or, preferably, elevated temperature for a period of time until hydrolysis is complete (e.g. 5 hours);
(viii) for compounds of formula I in which Y¹ and/or, if present, Y^{1a} represents —C(O)OR^{9a}, and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula I to be prepared),
   under standard conditions in the presence of the appropriate alcohol of formula XIII,

R^{9za}OH XIII

in which R^{9za} represents R^{9a} provided that it does not represent H, for example further in the presence of acid (e.g. concentrated H₂SO₄) at elevated temperature, such as at the reflux temperature of the alcohol of formula XIII;
(ix) for compounds of formula I in which Y¹ and/or, if present, Y^{1a} represents —C(O)OR^{9a}, in which R^{9a} is other than H, reaction of a compound of formula XIV,

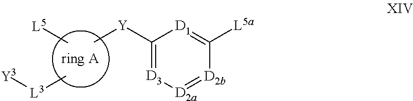

wherein at least one of L⁵ and L^{5a} represents an appropriate alkali metal group (e.g. sodium, potassium or, especially, lithium), a —Mg-halide, a zinc-based group or a suitable leaving group such as halo, —B(OH)₂, —B(OR^{wx})₂ or —Sn(R^{wx})₃ (in which each R^{wx} is as hereinbefore defined; and e.g. in the case of —B(OR^{wx})₂, the respective R^{wx} groups may be linked together to form a 4- to 6-membered cyclic group, such as a 4,4,5,5-tetramethyl-1,3,2-dioxaborolan-2-yl group), and the other may represent —Y¹ or —Y^{1a} (or hydrogen; as appropriate), and ring A, D₁, D₂ₐ, D_{2b}, D₃, Y, L³ and Y³ are as hereinbefore defined (the skilled person will appreciate that the compound of formula XIV in which L⁵ and/or L^{5a} represents an alkali metal (e.g. lithium), a Mg-halide or a zinc-based group may be prepared from a corresponding compound of formula XIV in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XV,

L⁶-Y^{b} XV

wherein Y^{b} represents —C(O)OR^{9a}, in which R^{9a} is other than H, and L⁶ represents a suitable leaving group known to those skilled in the art, such as halo (especially chloro or bromo), for example, the compound of formula XV may be Cl—C(O)OR^{9a}. Optionally, the reaction may be performed in the presence of a suitable additive known to the skilled person, e.g. Cu₂O or benzoquinone (as for example when the reaction is performed catalytically in the presence of boron or tin reagents, then a co-oxidant may be required). The reaction may be performed under standard reaction conditions, for example in the presence of a polar aprotic solvent (e.g. THF or diethyl ether);
(x) for compounds of formula I in which Y¹ and/or, if present, Y^{1a} represent —C(O)OR^{9a} in which R^{9a} is H, reaction of a compound of formula XIV as hereinbefore defined but in which L⁵ and/or L^{5a} (as appropriate) represents either:
(I) an alkali metal (for example, such as one defined in respect of process step (ix) above); or
(II) —Mg-halide,
   with carbon dioxide, followed by acidification under standard conditions known to those skilled in the art, for example, in the presence of aqueous hydrochloric acid;
   (xi) for compounds of formula I in which Y¹ and/or, if present, Y^{1a} represent —C(O)OR^{6a}, reaction of a corresponding compound of formula XIV as hereinbefore defined but in which L⁵ and/or L^{5a} (as appropriate) is a suitable leaving group known to those skilled in the art (such as a sulfonate group (e.g. a triflate) or, preferably, a halo (e.g. bromo or iodo) group; alternatively the leaving groups that L⁵ and/or L^{5a} may represent include —B(OH)₂, —B(OR^{wx})₂ or —Sn(R^{wx})₃, in which case the reaction may be performed in the presence of a suitable additive, such as one described in respect of process step (ix) above) with CO (or a reagent that is a suitable source of CO (e.g. Mo(CO)₆ or CO₂(CO)₈)), in the presence of a compound of formula XVI,

R^{9a}OH XVI

wherein R^{9a} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;
(xia) for compounds of formula I in which Y¹ and/or, if present, Y^{1a} represent —C(O)OH, reaction of a compound of formula XVIA,

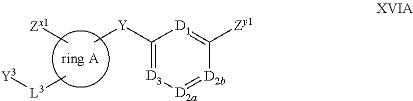

wherein at least one of Z^{x1} and Z^{y1} represents —CN, and the other may also represent —CN, or —Y¹ or —Y^{1a} (or hydrogen; as appropriate), and ring A, Y¹, Y^{1a}, D₁, D₂ₐ, D_{2b}, D₃, Y, L³ and Y³ are as hereinbefore defined, with water (i.e. hydrolysis);
(xii) for compounds of formula I in which Y represents —C≡C—, reaction of either a compound of formula XVII or XVIII,

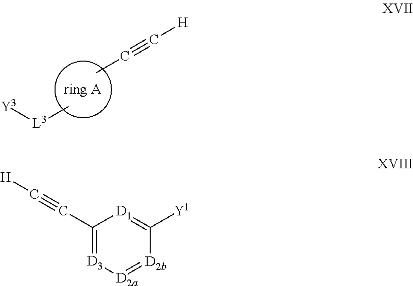

respectively with a compound of formula XIX or XX,

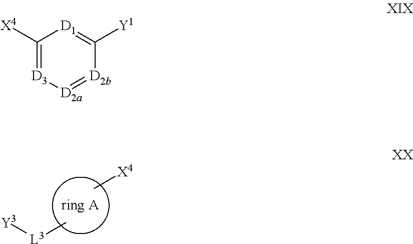

or protected derivatives thereof (e.g. an amino-protected derivative) wherein (in all cases) X⁴ represents a suitable leaving group such as one hereinbefore defined in respect of Z^{X} and Z^{y} and preferably represents chloro, bromo, or more preferably iodo, and ring A, D₁, D₂ₐ, D_{2b}, D₃, Y¹, L³ and Y³ are as hereinbefore defined, in the presence of an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like; and a metal halide, for example copper iodide) and a suitable base (for example sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof, and an appropriate solvent (e.g. tetrahydrofuran, pyridine, toluene, dichloromethane, chloroform, acetonitrile, dimethylformamide, dimethylsulfoxide, trifluoromethylbenzene, dioxane or triethylamine), performed at around room temperature or above (e.g. up to 40-180° C.), under conditions known to those skilled in the art. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof);
(xiii) for compounds of formula I in which Y represents —CH═CH—, either:
(a) reaction of either a compound of formula XXI or XXII,

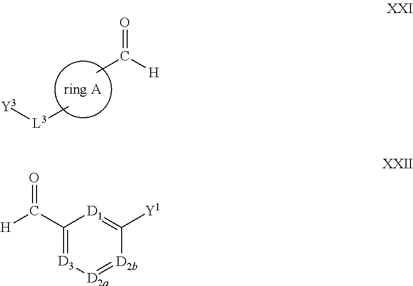

respectively with a compound of formula XXIII or XXIV,

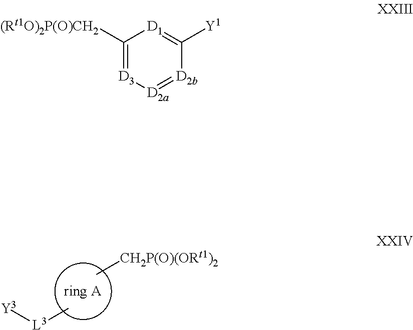

or protected derivatives thereof (e.g. amino-protected derivatives) wherein (in all cases) R¹¹ represents, at each occurrence when used herein, C₁₋₆ alkyl optionally substituted by one or more halo atoms, and ring A, D₁, D₂ₐ, D_{2b}, D₃, Y¹, L³ and Y³ are as hereinbefore defined, under standard Horner-Wadsworth-Emmons, reaction conditions, as appropriate;
(b) reaction of either a compound of formula XXI or XXII as hereinbefore defined, respectively with a compound of formula XXV or XXVI,

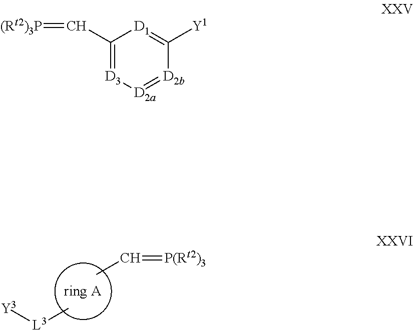

or protected derivatives thereof (e.g. amino-protected derivatives), wherein (in all cases) R^{t2} represents, at each occurrence when used herein, aryl (optionally substituted as hereinbefore defined by the integer B; e.g. unsubstituted phenyl, so forming for example a —P(Ph)₃ moiety) or C₁₋₆ alkyl optionally substituted by one or more halo atoms, and ring A, D₁, D₂ₐ, D_{2b}, D₃, Y¹, L³ and Y³ are as hereinbefore defined, under standard Wittig reaction conditions, as appropriate;
(c) reduction of a corresponding compound of formula I in which Y represents —C≡C—, in the presence of suitable reducing conditions, for example by catalytic hydrogenation in the presence of a suitable catalyst (to prevent reduction of the double bond formed as an intermediate; e.g. Lindlar's catalyst);
(d) reaction of either a compound of formula XXVIA or XXVIB,

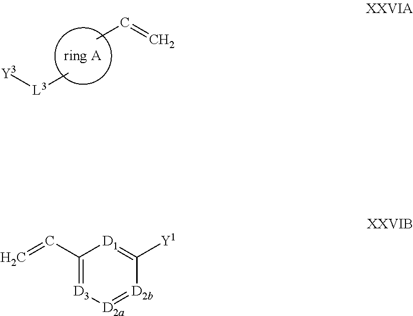

respectively with a compound of formula XXVIC or XXVID,

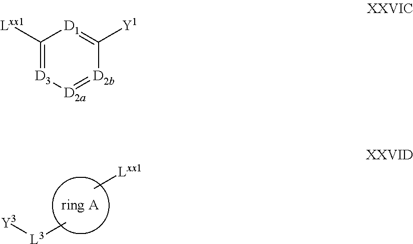

or protected derivatives thereof (e.g. amino-protected derivatives) wherein (in all cases) L^{xx1} represents a suitable leaving group such as one hereinbefore defined in respect of Z^{x} and Z^{y} (e.g. chloro, bromo iodo or a sulfonate group), and ring A, D₁, D₂ₐ, D_{2b}, D₃, Y¹, L³ and Y³ are as hereinbefore defined, under standard Heck reaction conditions, for example in accordance with the conditions hereinbefore described in respect of process (i)(B);
   (xiv) for compounds of formula I in which Y represents —CH₂—CH₂—, reduction of a corresponding compound of formula I in which Y represents —C≡C— or —CH═CH—, under standard conditions, for example under catalytic hydrogenation reaction conditions (for example in the presence of a precious metal catalyst, e.g. Pt or, preferably, Pd);
   (xv) for compounds of formula I in which Y represents —N(R^{f1})C(O)—, —C(O)—N(R^{g1})—, —N(Rⁿ¹)—S(O)₂— or —S(O)₂—N(R^{p1}), reaction of either a compound of formula XXVII or XXVIII,

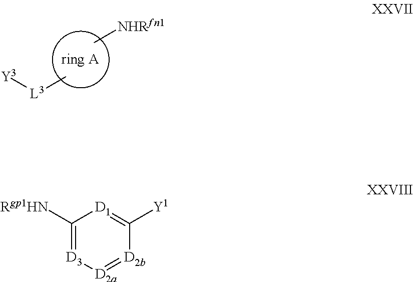

wherein R^{fn1} represents R^{f1} or Rⁿ¹ (as appropriate) and R^{gp1} represents R^{g1} or R^{p1} (as appropriate), respectively with a compound of formula XXIX or XXX,

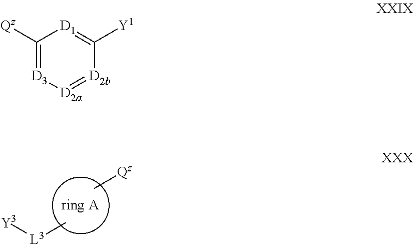

or protected derivatives thereof (e.g. amino-protected derivatives, or carboxylic acid protected derivatives), wherein Q^{z} represents, —C(O)L^{xx} or —S(O)₂L^{x1} (in which L^{xx} and L^{X1} independently represent —OH or a leaving group, such as halo, e.g. chloro), as appropriate, and ring A, D₁, D₂ₐ, D_{2b}, D₃, R^{f1}, R^{g1}, Rⁿ¹, R^{p1}, Y¹, L³ and Y³ are as hereinbefore defined (and Y¹ preferably represents —C(O)OR^{9a} in which R^{9a} is other than hydrogen and/or may be suitably protected with a standard protecting group), under standard coupling reaction conditions, for example (e.g. when L^{xx} and/or L^{x1} represent —OH) in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytris-pyrrolidinophosphonium hexafluoro-phosphate, bromo-tris-pyrrolidinophosphonium hexafluorophosphate, 2-(1H-benzotriazol-1-yl)-1,1,3,3-tetramethyluronium tetra-fluorocarbonate, 1-cyclohexyl-carbodiimide-3-propyloxymethyl polystyrene, O-(7-azabenzotriazol-1-yl)-N,N,N′,N′-tetramethyluronium hexafluorophosphate and/or O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof), an appropriate solvent (e.g. tetrahydrofuran, pyridine, toluene, dichloromethane, chloroform, acetonitrile, dimethylformamide, trifluoromethylbenzene, dioxane or triethylamine) and a further additive (e.g. 1-hydroxybenzotriazole hydrate). Alternatively (e.g. when L^{xx} and/or L^{x1} represent a suitable leaving group, such as chloro; which compounds may be prepared by converting the carboxylic acid group under standard conditions to the corresponding acyl chloride, e.g. in the presence of SOCl₂ or oxalyl chloride), the relevant acyl or sulfonyl chloride may be reacted with either a compound of formula XXVII or XXVIII respectively, for example under similar conditions to those mentioned above;
(xvi) for compounds of formula I in which Y represents —C(R^{k1})(R^{k2})—N(R^{m1})— or —N(Rⁱ¹)—C(R^{j1})(R^{j2})—, either:
(a) for those compounds in which R^{k1}, R^{k2}, R^{m1}, Rⁱ¹, R^{j1} and R^{j2} represent hydrogen, reaction of a compound of formula XXVII or XXVIII in which R^{fn1} and R^{gn1} both represent hydrogen, respectively with a compound of formula XXXI or XXXII,

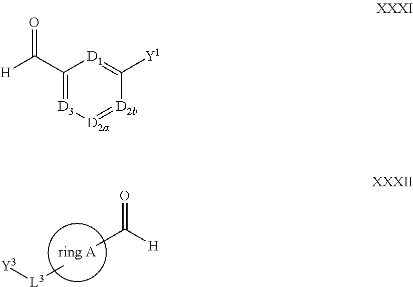

or protected derivatives thereof (e.g. amino-protected derivatives), wherein ring A, D₁, D₂ₐ, D_{2b}, D₃, Y¹, L³ and Y³ are as hereinbefore defined, for example in the presence of a chemoselective reducing agent such as sodium triacetoxyborohydride or sodium cyanoborohydride, or alternatively, as a two-step process included condensation and then reduction, which reduction step in this instance may be performed in the presence of a stronger reducing agent such as sodium borohydride or LiAlH₄ (in certain instances, the use of protecting groups may be essential);
(b) reaction of a compound of formula XXVII or XXVIII, respectively with a compound of formula XXXIA or XXXIIA,

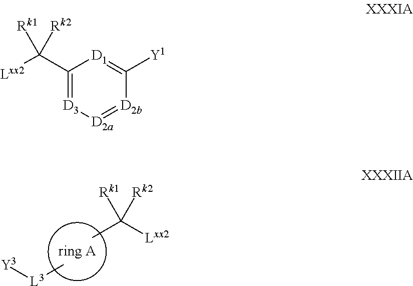

or protected derivatives thereof (e.g. amino-protected derivatives), wherein L^{xx2} represents a suitable leaving group such as one hereinbefore defined in respect of L^{xx1}, ring A, D₁, D₂ₐ, D_{2b}, D₃, Y¹, L³ and Y³ are as hereinbefore defined, and R^{k1} and R^{k2} are as hereinbefore defined (but at least one of these preferably represents hydrogen), for example under standard reaction conditions such as those hereinbefore described in respect of process step (i)(C);
(c) reaction of either a compound of formula XXXIII or XXXIV,

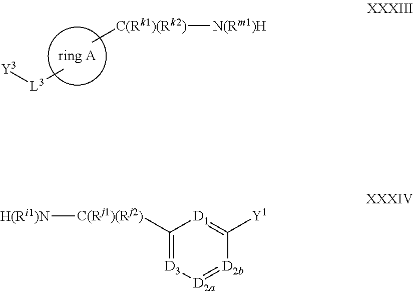

respectively with a compound of formula XIX or XX, as hereinbefore defined, or protected derivatives thereof, wherein ring A, D₁, D₂ₐ, D_{2b}, D₃, Y¹, R^{j1}, R^{j2}, R^{k1}, R^{k2}, Rⁱ¹, R^{m1}, L³ and Y³ are as hereinbefore defined, under reaction conditions known to those skilled in the art, for example such as those hereinbefore described in respect of process step (i)(B);
   (xvii) for compounds of formula I in which Y represents —O—C(R^{d1})(R^{d2})— or —C(R^{e1})(R^{e2})—O—, reaction of either a compound of formula XXXV or XXXVI,

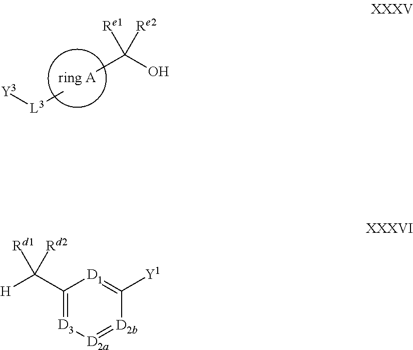

respectively with a compound of formula XIX or XX, as hereinbefore defined, or protected derivatives thereof, wherein ring A, D₁, D₂ₐ, D_{2b}, D₃, Y¹, R^{d1}, R^{d2}, R^{e1}, R^{e2}, L³ and Y³ are as hereinbefore defined, under reaction conditions known to those skilled in the art, for example such as those hereinbefore described in respect of process step (i)(B);
(xviii) for compounds of formula I in which Y represents —CH(OH)—, reduction of a compound of formula XXXVII,

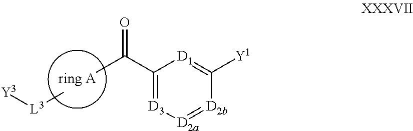

or protected derivatives thereof, wherein ring A, D₁, D₂ₐ, D_{2b}, D₃, Y¹, L³ and Y³ are as hereinbefore defined, in the presence of suitable reducing conditions, for example by reaction in the presence of e.g. NaBH₄ or NaBH₃CN (or the like), under conditions known to those skilled in the art;
(xix) for compounds of formula I in which Y represents a direct bond, reaction of a compound of formula XXXVIII or XXXIX,

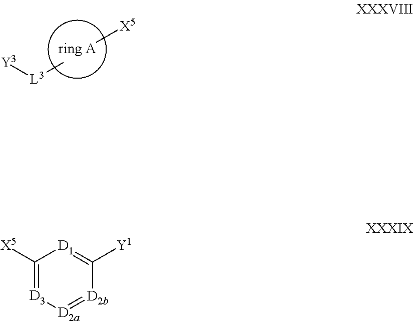

respectively with a compound of formula XIX or XX, as hereinbefore defined, wherein X⁵ represents a group such as one hereinbefore defined in respect of Z^{x} and Z^{y}, or metal halide (for example a zinc halide (e.g. —ZnCl), or a magnesium halide (e.g. —MgBr)), a stannane (e.g. —SnBu₃), an organoboronic acid (e.g. —B(OH)₂), or an organosilane (e.g. —Si(OEt)₃), and in which the groups X⁴ and X⁵ are mutually compatible, and ring A, D₁, D₂ₐ, D_{2b}, D₃, Y¹, L³ and Y³ are as hereinbefore defined, under standard reaction conditions (i.e. metal-catalysed chemistry), for example in the presence of a suitable base as hereinbefore described, performed at ambient temperature or above (e.g. up to about 40 to 180° C.);
(xx) for compounds of formula I in which R^{9a} represents hydrogen, formylation of a compound of formula XL,

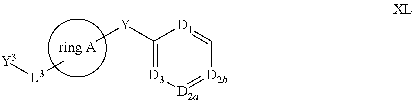

for example in the presence of suitable reagents such as P(O)Cl₃ and DMF, followed by oxidation under standard conditions;
(xxi) for compounds of formula I in which L² or L³ represent —N(H)—CH₂—, reductive amination of a compound of formula II as hereinbefore defined, with a compound of formula XLI,

Y^{a}—C(O)H XLI

wherein Y^{a} is as hereinbefore defined, under standard conditions, for example in the presence of a chemoselective reducing agent such as sodium triacetoxyborohydride or sodium cyanoborohydride, or alternatively, as a two-step process included condensation and then reduction, which reduction step in this instance may be performed in the presence of a stronger reducing agent such as sodium borohydride or LiAlH₄ (in certain instance, the use of protecting groups may be essential);
(xxii) reaction of a compound of formula XL, as hereinbefore defined, with a suitable reagent such as phosgene or triphosgene in the presence of a Lewis acid, followed by reaction in the presence of a compound of formula XVI as hereinbefore defined (i.e. a hydrolysis or alcoholysis reaction);
(xxiii) for compounds of formula I in which L² and/or L³ represent —OA¹⁷-, reaction of a compound of the formula XLII,

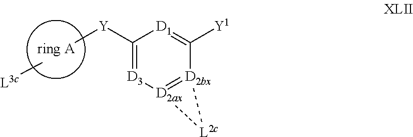

or a protected derivative thereof (e.g. an amino-protected derivative), wherein one of D₂ₐₓ and D_{2bx} represents D₂ and the other represents —C(-L^{2c})= (i.e. the L^{2c} substituent is attached to either one of D₂ₐₓ and D_{2bx}), L^{2c} represents —OH or -L²-Y², L^{3c} represents —OH or -L³-Y³, provided that at least one of L^{2c} and L^{3c} represents —OH, and Y, ring A, D₁, D₂, D₃ and Y¹ are as hereinbefore defined, with:
(a) when A¹⁷ represents a direct bond, a compound of formula XLIII,

Y^{c}—X^{c} XLIII
wherein Yd represents Y² or Y³ (as appropriate/required) as hereinbefore defined, and X^{c} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group), for example under reaction conditions described hereinbefore in respect of process step (i)(B) above;
(b) when A¹⁷ represents —CH₂—, with a compound of formula XLIV,

Y^{c}—CH₂—X^{d} XLIV
wherein X^{d} represents a suitable leaving group such as chloro, bromo, iodo or a sulfonate group (e.g. —OS(O)₂CF₃, —OS(O)₂CH₃, —OS(O)₂PhMe or a nonaflate), and Y^{c} is as hereinbefore defined, for example under reaction conditions described hereinbefore in respect of process step (i)(C) above;
   (xxiv) for compounds of formula I in which Y represents —C(OR^{d1})(R^{h1})— and R^{q1} is other than hydrogen, reaction of a corresponding compound of formula I in which R^{q1} is hydrogen, with either:
(a) a compound of formula XLIVA,

R^{q1a}—OH XLIVA
wherein R^{q1a} represents R^{q1} provided that it does not represent hydrogen (i.e. it represents an alkyl group as hereinbefore defined, preferably in which there is no ═O or alkoxy substituent a to the oxygen atom), for example under acidic reaction conditions, e.g. in the presence of CF₃COOH or HCl);
(b) an appropriate acylating reagent (when there is a ═O substituent a to the oxygen atom) or an appropriate sulfonylating reagent (when R^{q1a} is —S(O)₂R^{r1}).

Compounds of formula II (or protected, e.g. mono-protected derivatives thereof) may be prepared by reduction of a compound of formula XLV,

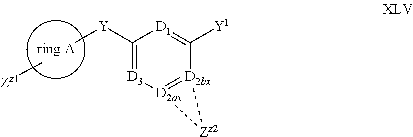

or a protected derivative thereof (e.g. an amino-protected derivative) wherein one of D₂ₐₓ and D_{2bx} represents D₂ and the other represents —C(—Z^{z2})═ (i.e. the Z^{z2} substituent is attached to either one of D₂ₐₓ and D_{2bx}), Z^{z1} represents —N₃, —NO₂, -L³-Y³ or a protected —NH₂ group, Z^{z2} represents —N₃, —NO₂, -L²-Y² or a protected —NH₂ group, provided that at least one of Z^{z1} and Z^{z2} represents —N₃ or —NO₂, and ring A, D₁, D₂, D₃, Y and Y¹ are as hereinbefore defined, under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane). Further, azides may also be reduced by e.g. phosphines (such as PPh₃).

Compounds of formula II in which both L^{2a} and L^{3a} represent —NH₂ (or protected derivatives thereof) may also be prepared by reaction of a compound of formula IX as hereinbefore defined, with ammonia, or preferably with a protected derivative thereof (e.g. benzylamine or Ph₂C═NH), under conditions such as those described hereinbefore in respect of preparation of compounds of formula I (process step (iii) above).

Compounds of formulae II or IX in which Y¹ represents —C(O)OR^{9a}, may be prepared by:

(I) reaction of a compound of formula XLVI,

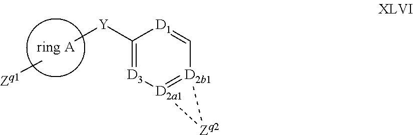

wherein Z^{q1} and Z^{q2} respectively represent Z^{x} and Z^{y} (in the case of preparation of compounds of formula IX) or L^{2a} and L^{3a} (in the case of preparation of compounds of formula III), D₂ₐ₁ and D_{2b1} respectively represent D₂ₐₓ and D_{2bx} (in the case of preparation of compounds of formula III) or D_{2az} and D_{2bz} (in the case of preparation of compounds of formula IX) and ring A, D₁, D₂ₐₓ, D_{2bx}, D_{2az}, D_{2bz}, D_{2bz}, D₃, L^{2a}, L^{3a}, Z^{x} and Z^{y} are as hereinbefore defined, with a suitable reagent such as phosgene or triphosgene in the presence of a Lewis acid, followed by reaction in the presence of a compound of formula XVII as hereinbefore defined, hence undergoing a hydrolysis or alcoholysis reaction step (the skilled person will appreciate that in certain instances, the use of protecting groups using these conditions may be essential, e.g. to protect an amino group);
(II) for such compounds in which R^{9a} represents hydrogen, formylation of a compound of formula XLVI as hereinbefore defined, for example in the presence of suitable reagents such as P(O)Cl₃ and DMF, followed by oxidation under standard conditions (the skilled person will appreciate that in certain instances, the use of protecting groups using these conditions may be essential, e.g. to protect an amino group);
(III) reaction of a compound of formula XLVII,

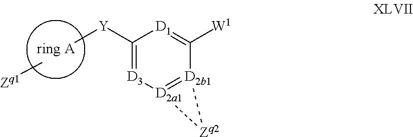

wherein W¹ represents a suitable leaving group such as one defined by Z^{x} and Z^{y} above, and ring A, D₁, D₂ₐ₁, D_{2b1}, D₃, Y, Z^{q1} and Z^{q2} are as hereinbefore defined, are as hereinbefore defined, with CO (or a reagent that is a suitable source of CO (e.g. Mo(CO)₆ or CO₂(CO)₈) followed by reaction in the presence of a compound of formula XVI as hereinbefore defined, under reaction conditions known to those skilled in the art, for example such as those hereinbefore described in respect of preparation of compounds of formula I (process step (i)(A)(b) or (i)(B) above), e.g. the carbonylation step being performed in the presence of an appropriate precious metal (e.g. palladium) catalyst;
(IV) reaction of a compound of formula XLVIII,

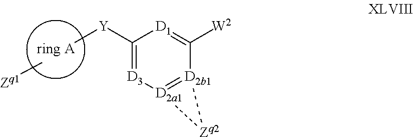

wherein W² represents a suitable group such as an appropriate alkali metal group (e.g. sodium, potassium or, especially, lithium), a —Mg-halide or a zinc-based group, and ring A, D₁, D₂ₐ₁, D_{2b1}, D₃, Y, Z^{q1} and Z^{q2} are as hereinbefore defined, with e.g. CO₂ (in the case where R^{9a} in the compounds to be prepared represents hydrogen), under reaction conditions known to those skilled in the art. The skilled person will appreciate that this reaction step may be performed directly after (i.e. in the same reaction pot) the preparation of compounds of formula XLVIII (which is described hereinafter).

Compounds of formula II in which L^{3a} represents —NH₂, which is α to a —Y^{1a} group present, which represents —C(O)OH, reaction of a compound of formula XLIX,

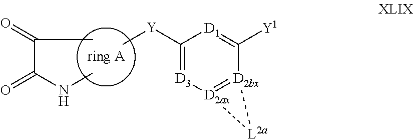

wherein ring A, D₁, D₂ₐₓ, D_{2bx}, D₃, Y, L^{2a} and Y¹ are as hereinbefore defined under standard reaction conditions, for example such as those described in Sheibley, F. E. and McNulty, J. S. J. Org. Chem., 1956; 21, 171-173, e.g. in the presence of H₂O₂, which is preferably in the presence of an alkaline solution.

Alternatively still, compounds of formula II in which D₂ₐₓ represents D₂ₐ, D_{2bx} represents —C(-L^{2a})=, and L^{2a} represents —NH₂, may be prepared by reaction of a compound of formula L,

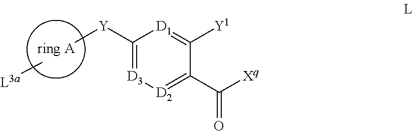

wherein X^{q} represents —OH, —NH₂ or —N₃, and L^{3a}, Y, D₁, D₂, D₃ and ring A are as hereinbefore defined, under standard reaction conditions, for example:
(i) when X^{q} represents —OH, under Schmidt reaction conditions, or variants thereof, in the presence of HN₃ (which may be formed in by contacting NaN₃ with a strong acid such as H₂SO₄). Variants include reaction with diphenyl phosphoryl azide ((PhO)₂P(O)N₃) in the presence of an alcohol (such as tert-butanol; thereby forming a t-Boc protected derivative of formula XL) which may result in the formation of a carbamate intermediate;
(ii) when X^{q} represents —NH₂, under Hoffmann rearrangement reaction conditions, for example in the presence of NaOBr (which may be formed by contacting NaOH and Br₂) which may result in the formation of a carbamate intermediate;
(iii) when X^{q} represents —N₃ (which compound itself may be prepared from the corresponding hydrazide under standard diazotization reaction conditions, e.g. in the presence of NaNO₂ and a strong acid such as H₂SO₄ or HCl), under Curtius rearrangement reaction conditions, which may result in the formation of an intermediate isocyanate (or a carbamate if treated with an alcohol),
all of which may be followed by, if necessary (e.g. if the formation of the free amine is desired), hydrolysis, for example in the presence of water and base (e.g. one hereinbefore described in respect of process step (vii) above) when a lower alkyl carbamate (e.g. methyl or ethyl carbamate) is formed as an intermediate or under acidic conditions when e.g. a tert-butyl carbamate is formed as an intermediate, or, when a benzyl carbamate intermediate is formed, under hydrogenation reaction conditions (e.g. catalytic hydrogenation reaction conditions in the presence of a precious metal catalyst such as Pd).

Compounds of formula VIII may be prepared by reaction of a corresponding compound of formula II in which L^{2a} or L^{3a} (as appropriate) represent —NH₂, with phosgene or triphosgene, for example in the presence of a suitable base (e.g. one hereinbefore defined in respect of preparation of compounds of formula I (e.g. triethylamine). When the compound of formula VIII is synthesised accordingly, it need not be isolated and/or purified when further employed in the synthesis of a compound of formula I (see process step (i) above).

Compounds of formula IX in which Z^{x} and Z^{y} represent a sulfonate group may be prepared from corresponding compounds in which the Z^{x} and Z^{y} groups represent a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i)(C), e.g. an aqueous solution of K₃PO₄ in toluene) preferably at or below room temperature (e.g. at about 10° C.).

Compounds of formula XVIA may be prepared by reaction of a compound of formula XLIXA,

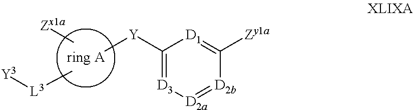

wherein at least one of Z^{x1a} and Z^{y1a} represents a suitable leaving group as hereinbefore defined by Z^{x} and Z^{y}, and the other may also represent such a leaving group or —Y¹ or —Y^{1a} (or hydrogen; as appropriate), and ring A, Y¹, Y^{1a}, D₁, D₂ₐ, D_{2b}, D₃, Y, L³ and Y³ are as hereinbefore defined, with an appropriate reagent for the introduction of a cyano group, such as Zn(CN)₂, under standard conditions, for example, such as those hereinbefore described in respect of preparation of compounds of formula I (process step (i)(B) above).

Compounds of formulae XVII and XVIII may be prepared by reaction of a compound of formula XIX or XX, with a compound of formula LI,

HC≡C-L^{c} LI

wherein L^{c} represents a suitable protecting group, for example a silane (e.g. trimethyl silane) under reaction conditions known to those skilled in the art, for example such as those described in respect of process step (i)(B) above, followed by an appropriate reaction known to those skilled in the art for the removal of the protecting group (for example reaction in the presence of a fluoride salt, (e.g. a tetraalkylammonium fluoride such as tetrabutylammonium fluoride)).

Compounds of formula XXXVII may be prepared by oxidation of a compound of formula LIA,

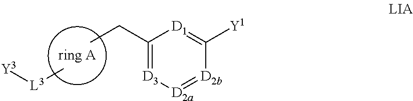

wherein ring A, D₁, D₂, D₃, Y¹, L², Y², L³ and Y³ are as hereinbefore defined, in the presence of a suitable oxidising agent, for example, KMnO₄, optionally in the presence of a suitable solvent, such as acetone, and an additive such as magnesium sulfate.

Compounds of formula XXXVII may be prepared by reaction of either a compound of formula LIB or LIC,

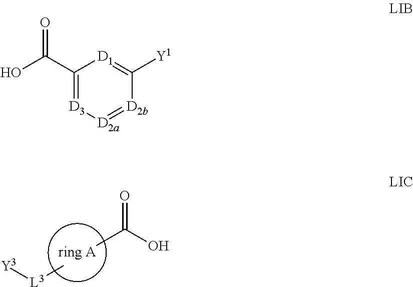

respectively with a compound of formula LID or LIE,

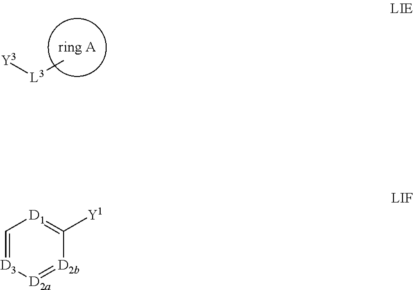

wherein (in all cases) ring A, D₁, D₂ₐ, D_{2b}, D₃, Y¹, L², Y², L³ and Y³ are as hereinbefore defined, in the presence of a suitable reagent that converts the carboxylic acid group of the compound of formula LIC or LID to a more reactive derivative (e.g. an acid chloride or acid anhydride, or the like) such as POCl₃, in the presence of ZnCl₂, for example as described in Organic and Biomolecular Chemistry (2007), 5(3), 494-500 or, more preferably, PCl₃, PCl₅, SOCl₂ or (COCl)₂. Alternatively, such a reaction may be performed in the presence of a suitable catalyst (for example a Lewis acid catalyst such as SnCl₄), for example as described in Journal of Molecular Catalysis A: Chemical (2006), 256(1-2), 242-246 or under alternative Friedel-crafts acylation reaction conditions (or variations thereupon) such as those described in Tetrahedron Letters (2006), 47(34), 6063-6066; Synthesis (2006), (21), 3547-3574; Tetrahedron Letters (2006), 62(50), 11675-11678; Synthesis (2006), (15), 2618-2623; Pharmazie (2006), 61(6), 505-510; and Synthetic Communications (2006), 36(10), 1405-1411. Alternatively, such a reaction between the two relevant compounds may be performed under coupling reaction conditions (e.g. Stille coupling conditions), for example as described in Bioorganic and Medicinal Chemistry Letters (2004), 14(4), 1023-1026.

Compounds of formula LIA may be prepared by reaction of a compound of formula LIE with a compound of formula LIF, both as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.) Preferably, the formaldehyde is added (e.g. slowly) to an acidic solution of the compound of formula LIE at about 50° C., with the reaction temperature rising to about 70° C. after addition is complete. When acidic conditions are employed, precipitation of the compound of formula LIA may be effected by the neutralisation (for example by the addition of a base such as ammonia).

Compounds of formula XLVIII may be prepared in several ways. For example, compounds of formula XLVIII in which W² represents an alkali metal such as lithium, may be prepared from a corresponding compound of formula XLVI (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamino group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium coordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XLVIII may be prepared by reaction of a compound of formula XLVII in which W¹ represents chloro, bromo or iodo by a halogen-lithium reaction in the presence of an organolithium base such as t- or n-butyllithium under reaction conditions such as those described above. Compounds of formula XLVIII in which W² represents —Mg-halide may be prepared from a corresponding compound of formula XLVII in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XLVIII in which W² represents a zinc-based group (e.g. using ZnCl₂).

Compounds of formula XLIX may be prepared by reaction of a compound of formula LII,

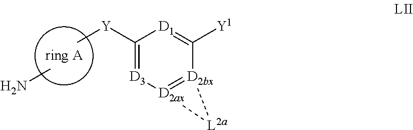

wherein ring A, D₁, D₂ₐₓ, D_{2bx}, D₃, L^{2a}, Y and Y¹ are as hereinbefore defined, with chloral hydrate, hydroxylamine hydrochloride, sodium sulfate and hydrochloric acid, followed by reaction in the presence of concentrated sulfuric acid, for example as described in the Sheibley et al journal article referenced herein.

Compounds in which Y¹ represents —C(O)OH may be prepared by hydrolysis of a corresponding compound in which there is a cyano group present. Compounds in which there is a cyano group attached to an aromatic ring may be prepared by standard nucleophilic aromatic substitution of a corresponding compound in which a leaving group such as fluoro is present, under standard reaction conditions, e.g. under palladium catalysed cyanation reaction conditions.

Compounds other than compounds of formula I may be commercially available, known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents D₁, D₂ₐ, D_{2b}, D₃, Y¹, L³ and Y³ (as well as L² and Y²) in final compounds of the invention or relevant intermediates may be modified one or more times, after or during the processes described above by way of methods that are well known to those skilled in the art. Examples of such methods include substitutions, reductions, oxidations, alkylations, acylations, sulfonylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ (or, if present, Y^{1a}) represents —C(O)OR^{9a} in which R^{9a} does not initially represent hydrogen (so providing at least one ester functional group), the skilled person will appreciate that at any stage during the synthesis (e.g. the final step), the relevant R^{9a}-containing group may be hydrolysed to form a carboxylic acid functional group (i.e. a group in which R^{9a} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include the reduction of a nitro group to an amino group, the hydrolysis of a nitrile group to a carboxylic acid group, and standard nucleophilic aromatic substitution reactions, for example in which a fluoro- or bromo-phenyl group is converted into a cyanophenyl group by employing a source of cyanide ions (e.g. KCN) as a reagent (alternatively, in this case, palladium catalysed cyanation reaction conditions may also be employed).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing hetereocycles may also be performed with compounds that are pre-cursors to heterocycles, and which pre-cursors may be converted to those heterocycles at a later stage in the synthesis.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an α-carboxy group (i.e. such that the cyclic moiety is formed between the two functional groups, for example as described hereinafter in the formation of intermediate (I)).

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without all of the provisos except (A)(I)(a), (A)(I)(d) and (B)(II) (i.e. but without provisos (A)(I)(b), (A)(I)(c), (A)(I)(e), (A)(II), (B)(I) and (C)), for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which Y¹ (or, if present, Y^{1a}) represents —C(O)OR^{9a} in which R^{9a} is/are other than hydrogen, so forming an ester group) may possess no or minimal pharmacological activity as such, but may be administered parenterally or orally, and thereafter be metabolised in the body to form compounds of the invention that possess pharmacological activity as such (including, but not limited to, corresponding compounds of formula I, in which Y¹ (or, if present, Y^{1a}) represents —C(O)OR^{9a} in which R^{9a} represent hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III (preferably MGST-II and/or MGST-III), thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Where a condition has an inflammatory component associated with it, or a condition characterized by inflammation as a symptom, the skilled person will appreciate that compounds of the invention may be useful in the treatment of the inflammatory symptoms and/or the inflammation associated with the condition.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aeruginosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without all of the provisos except (A)(I)(a), A(I)(d) and B(II), in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined but without the provisos (e.g. but without all of the provisos except (A)(I)(a), (A)(I)(d) and (B)(II)), or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. thromboxane receptor (TP) antagonists, leukotriene receptor antagonists (LTRAs), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activating protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined but without the provisos (e.g. without all of the provisos except (A)(I)(a), A(I)(d) and B(II)); and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos (e.g. without all of the provisos except (A)(I)(a), A(I)(d) and B(II)), another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined but without the provisos (e.g. without all of the provisos except (A)(I)(a), A(I)(d) and B(II)), in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined but without all of the provisos except (A)(I)(a), A(I)(d) and B(II), or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

### In Vitro Assay

In the assay LTC₄ synthase catalyses the reaction where the substrate LTA₄ methyl ester is converted to the corresponding LTC₄ methyl ester. Recombinant human LTC₄ synthase is expressed in Piccia pastoralis and the purified enzyme is dissolved in 25 mM Tris-buffer pH 7.8 and stored at −20° C. The assay is performed in phosphate buffered saline (PBS) pH 7.4, supplemented with 5 mM glutathione (GSH). The reaction is terminated by addition of acetonitrile/MeOH/acetic acid (50/50/1). The assay is performed at rt in 96-well plates. Analysis of the formed LTC₄ methyl ester is performed with reversed phase HPLC (Waters 2795 utilizing an Onyx Monolithic C18 column). The mobile phase consists of acetonitrile/MeOH/H₂O (32.5/30/37.5) with 1% acetic acid pH adjusted with NH₃ to pH 5.6, and absorbance measured at 280 nm with a Waters 2487 UV-detector.

The following is added chronologically to each well:
1. 50 μL assay buffer, PBS with 5 mM GSH.
2. 0.5 μL inhibitor in DMSO.
3. 2 μL LTC₄ synthase in PBS. The total protein concentration in this solution is 0.025 mg/mL. Incubation of the plate at room temperature for 10 minutes.
4. 0.5 μL LTA₄ methyl ester. Incubation of the plate at rt for 1 min.
5. 50 μL stop solution.
80 μL of the incubation mixture is analysed with HPLC.

Alternatively HTRF detection of LTC₄ can be used:

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM Tris-buffer pH 7.8 supplemented with 0.1 mM glutathione (GSH) and stored at −80° C. The assay is performed in phosphate buffered saline (PBS) pH 7.4 and 5 mM GSH in 384-well plates.

The following is added chronologically to each well:

1. 48 μL LTC₄ synthase in PBS with 5 mM GSH. The total protein concentration in this solution is 0.5 μg/mL.
2. 1 μL inhibitor in DMSO (final concentration 10 μM).
3. Incubation of the plate at rt for 10 min.
4. 1 μL LTA₄ (final concentration 2.5 μM).
5. Incubation of the plate at rt for 5 min.
6. 10 μl of the incubation mixture is analysed using HTRF detection.

### EXAMPLES

The invention is illustrated by way of the following examples, in which the following abbreviations may be employed:

| | |
|---|---|
| aq | aqueous |
| atm | atmosphere |
| brine | saturated solution of NaCl in water |
| DMAc | N,N-dimethylacetamide |
| DMAP | N,N-dimethyl-4-aminopyridine |
| DMF | dimethylformamide |
| EtOAc | ethyl acetate |
| EtOH | EtOH |
| MeOH | MeOH |
| NMR | nuclear magnetic resonance |
| Pd/C | Palladium on charcoal |
| rt | room temperature |
| rx | reflux temperature |
| sat | saturated |
| THF | tetrahydrofuran |

Chemicals specified in the synthesis of the compounds in the examples were commercially available from, e.g. Sigma-Aldrich, Acros, Alfa Aesar, Menai Organics, Chembrige, Pfaltz & Bauer or Matrix Scientific.

### Preparation of Starting Materials and Active Inhibitors

### Synthesis of methyl-2-amino-5-nitrobenzoate

Concentrated sulfuric acid (7 mL) was added dropwise to a suspension of potassium 2-amino-5-nitrobenzoate (10 g, 45 mmol) in MeOH (150 mL). The mixture was heated at rx for 4 days. After cooling to rt, water was added and the mixture was neutralized with Na₂CO₃. The solids were collected and recrystallized from MeOH to afford 7 g (80%) of the subtitle compound.

### Synthesis of 5,5′-(hydroxymethylene)bis(2-(4-butylbenzamido)benzoic acid) (Ex 1)

### Step 1: Dimethyl 3,3′-methylenebis(6-aminobenzoate) (I)

Methyl 2-amino benzoate (15.1 g, 100 mmol) was added to water (126 mL) at 50° C. Concentrated HCl (26 mL) and formaldehyde (3% aq, 36 mL) were added in portions during 30 min. The mixture was stirred at 70° C. for 4.5 h. After cooling to rt, ammonia (aq, sat, 35 mL) was added to pH ˜8. The precipitate was collected, washed with water, dried and purified by chromatography, furnishing compound I (8.33 g, 53%).

### Step 2: Dimethyl 3,3′-methylenebis(6-acetamidobenzoate) (II)

Acetyl chloride (5.2 mL, 72.8 mmol) was added to a mixture of compound I (7.54 g, 24 mmol), triethylamine (10.08 mL, 72.8 mmol) and dioxane (160 mL) at 0° C. The mixture was stirred at it for 22 h, concentrated to a small volume and poured into water. The precipitate was collected, washed with water and dried to give compound II (9.09 g, 95%).

### Step 3: Dimethyl 3,3′-carbonylbis(6-acetamidobenzoate) (III)

KMnO₄ (12 g, 76 mmol) was added in portions to a stirred mixture of compound II (9.0 g, 22.5 mmol), MgSO₄ (15% aq, 20 mL) and acetone (500 mL). After 8 d at rt, the mixture was filtered through Celite and washed with CH₂Cl₂. The filtrate was washed with water, brine, MgSO₄ (aq, sat) and concentrated to afford 6.3 g (68%) of compound III.

### Step 4: Dimethyl 3,3′-carbonylbis(6-aminobenzoate) (IV)

A mixture of compound III (6.0 g, 14.5 mmol), MeOH (600 mL) and HCl (aq, 5 M, 540 mL) was stirred at rx for 1.5 h and concentrated to a smaller volume. NaHCO₃ was added to pH ˜7-8 and the mixture was extracted with EtOAc. The combined extracts were washed with brine and dried (MgSO₄) to afford compound IV. Yield 3.85 g (81%).

### Step 5: Dimethyl 5,5′-carbonylbis(2-(4-butylbenzamido)benzoate) (V)

Compound IV was aroylated according to the general method A, using 4-butylbenzoyl chloride, furnishing compound V.

### Step 6: Dimethyl 5,5′-(hydroxymethylene)bis(2-(4-butylbenzamido)benzoate) VI

A mixture of compound V (143 mg, 022 mmol, NaBH₄ (10 mg, 0.26 mmol), THF (10 mL) and water (1 mL) was stirred at rx for 1 h. The mixture was concentrated to a smaller volume, HCl (1 M, aq) was added and the mixture extracted with EtOAc. The combined extracts were washed with brine and dried (Na₂SO₄). Concentration gave compound VI (142 mg).

Hydrolysis according to general method B furnished the title compound (Example 1).

### Method A for Aroylation of Amines

A mixture of the appropriate amine (0.5 mmol), aroyl chloride (1.5 mmol) and toluene (10 mL) was heated at rx 4 h. The mixture was cooled and diluted with EtOAc. Extractive workup (NaHCO₃ (aq, sat) and brine) followed by drying (Na₂SO₄) and concentration gave a residue which was recrystallized to give the corresponding amides. Alternatively, MeOH was added to the reaction mixture which was stirred for 20 min, concentrated, followed by recrystallization of the residue.

### General Procedure B for Ester Hydrolysis

A mixture of the appropriate ester (0.15 mmol), NaOH (60 mg, 1.5 mmol), water (2 mL) and EtOH (10 mL) was heated at 60° C. for 0.5 h. After cooling to 0° C. and addition of HCl (1 M, aq) to pH ˜2, the precipitate was collected and recrystallized, delivering the corresponding free acids.

### Preparation of Symmetrical Bi-Aryl Compounds, Table 1:

### Example 1:2

A mixture of benzenesulfonyl chloride (17.6 g, 100 mmol), 4,4′-diaminobiphenyl-3,3′-dicarboxylic acid (VII) (2.72 g, 10 mmol) and Na₂CO₃ (aq, sat, 100 mL) was heated at rx for 0.5 h. Activated carbon was added and the mixture was filtered and the filtrate acidified with HCl (1 M, aq). The precipitate was collected, washed with MeOH and recrystallized from DMAc to give the title compound. Yield 300 mg (5.54%).

### Example 1:3

A mixture of compound VII (1.36 g, 5 mmol), 3-chlorobenzoyl chloride (2.50 g, 20 mmol) and DMAc (30 mL) was stirred for 24 h at rt. The precipitate was collected and washed consecutively with 10 mL DMAc and 10 mL MeOH. Recrystallization in an appropriate solvent gave the title compound. Yield 980 mg (35%).

### Example 1:4

A mixture of compound VII (250 mg, 0.918 mmol), 1-chloro-3-isocyanatobenzene (338 mg, 2.20 mmol) and THF (30 mL) was stirred at rt for 2 d. After concentration, the residue was recrystallized from THF/n-hexane to deliver the title compound. Yield 30 mg (6%).

### Example 1:5

Pyridine-3-sulfonyl chloride hydrochloride (1.71 g, 8 mmol) was added in small portions to a mixture of compound VII (0.544 g, 2 mmol) and sodium carbonate (aq, sat, 20 mL) and stirred at rt until nearly no monosulfonylation product was detected by LC-MS. The precipitate was collected and washed with water and MeOH and dried. After recrystallization from DMAc and MeOH, the title compound was obtained. Yield 130 mg (11%).

### Example 1:6

A mixture of compound VII (1.36 g, 5 mmol), 4-tert-butylbenzoyl chloride (3.93 g, 20 mmol) and DMAc (30 mL) was stirred for 24 h at rt. The precipitate was collected and washed consecutively with 10 mL DMAc and 10 mL MeOH. Recrystallization from DMAc gave the title compound. Yield 1.22 g (41.2%).

### Example 1:7

A mixture of compound VII (1.36 g, 5 mmol), 2-(4-chlorophenyl)acetyl chloride (3.78 g, 20 mmol) and DMAc (30 mL) was stirred for 24 h at rt. The precipitate was collected and washed consecutively with 10 mL DMAc and 10 mL MeOH. Crystallization from DMAc gave the target compound. Yield 1.84 g (63.7%).

TABLE 1

| No | Title Compound |
|---|---|
| 1:2 | 4,4′-bis(phenylsulfonamido)biphenyl-3,3′-dicarboxylic acid |
| 1:3 | 4,4′-bis(3-chlorobenzamido)biphenyl-3,3′-dicarboxylic acid |
| 1:4 | 4,4′-bis(3-(3-chlorophenyl)ureido)biphenyl-3,3′-dicarboxylic acid |
| 1:5 | 4,4′-bis(pyridine-3-sulfonamido)biphenyl-3,3′-dicarboxylic acid |
| 1:6 | 4,4′-bis(4-tert-butylbenzamido)biphenyl-3,3′-dicarboxylic acid |
| 1:7 | 4,4′-bis(2-(4-chlorophenyl)acetamido)biphenyl-3,3′-dicarboxylic acid |

### Commercially Available Compounds:

4,4′-bis(2-aminobenzamido)biphenyl-3,3′-dicarboxylic acid (Example 1:8)
4,4′-bis(4-methylphenylsulfonamido)biphenyl-3,3′-dicarboxylic acid (Example 1:9)
4,4′-bis(1-naphthamido)biphenyl-3,3′-dicarboxylic acid (Example 1:10)
4,4′-bis(benzamido)biphenyl-3,3′-dicarboxylic acid (Example 1:11)
4,4′-bis(4-acetoxybenzamido)biphenyl-3,3′-dicarboxylic acid (Example 1:12)

### Two Carbon Linker Compounds, Table 2

Step 1: A mixture of 2-amino-5-iodobenzoic acid (3.0 g, 11.4 mmol), aroyl chloride (33.9 mmol) and pyridine was stirred at rt for 3 h. After concentration, water was added and the precipitate was collected and washed with water. The solid was dissolved in MeOH (20 mL) and dioxane (20 mL). Sodium methoxide (1.44 g, 26.6 mmol) was added and the mixture stirred at rt for 2 h. The mixture was acidified to pH ˜1-2 with HCl (aq) and concentrated. Extractive workup (EtOAc, water, NaHCO₃ (aq, sat), brine), drying (Na₂SO₄) and concentration, afforded methyl 5-iodo-2-(arylamido)benzoate.

Step 2: A mixture of methyl 5-iodo-2-(arylamido)benzoate (2.5 mmol), PdCl₂(PPh₃)₂ (106 mg, 0.15 mmol), CuI (95 mg, 0.5 mmol), tetrabutylammonium iodide (1.39 g, 3.75 mmol), triethylamine (5.9 mL) and acetonitrile (60 mL) was stirred at rt for 5 min. Ethynyltrimethylsilane (0.71 mL, 5 mmol) was added and the mixture was stirred at rt for 1 h and poured into NH₄Cl (aq, sat). The precipitate was collected and washed with water. The residue was dried and dissolved in THF. Tetrabutylammonium fluoride (1 M in THF) was added slowly under stirring at 0° C. until all starting material was consumed. Concentration and extractive workup (EtOAc, water, brine), drying (Na₂SO₄) and concentration afforded methyl 5-ethynyl-2-(4-(arylamido)benzoate.

Step 3: A mixture of the appropriate 5-iodo-2-(arylamido)benzoate (prepared as described in Step 1) (0.63 mmol), PdCl₂(PPh₃)₂ (27 mg, 0.038 mmol), CuI (245 mg, 0.126 mmol), tetrabutylammonium iodide (0.9 mmol), triethylamine (1.5 mL) and acetonitrile (10 mL) was stirred at rt for 5 min. Methyl 5-ethynyl-2-(4-(arylamido)benzoate (0.63 mmol), prepared as described in Step 2, was added and the mixture was stirred at rt for 1 h and at 60° C. for 1 h. The mixture was poured into NH₄Cl (aq, sat). The precipitate was collected and washed with water. Purification by chromatography furnished the title compounds in Table 2.

Step 4: A mixture of methyl 2-(arylamido)-5-(arylethynyl)benzoate (0.12 mmol), Pd/C (10%, 20 mg), THF (10 mL) and EtOH (10 mL) was hydrogenated at ambient temperature an pressure for 2 h. The mixture was filtered through Celite and the solids washed with EtOH. Concentration of the combined filtrates gave a residue which was hydrolyzed according to general method B furnishing the title compounds in Table 2 (2:1-2:4).

Step 4 was not performed for example 2:5. In that case, hydrolysis was carried out directly after step 3.

TABLE 2

| | | | Yield |
|---|---|---|---|
| | | | (%) |
| No | Title Compound | Aryl iodide in Step 3 | Step 3 |
| 2:1 | 5-{2-[3-Carboxy-4- | Methyl 5-iodo- | 55 |
| | (4-methoxybenzoyl- | 2-(4-methoxy- | |
| | amino)phenyl]ethyl}- | benzamido)- | |
| | 2-(3-trifluoromethyl- | benzoate | |
| | benzoylamino)benzoic | | |
| | acid | | |
| 2:2 | 5-{2-[3-Carboxy-4- | Methyl 2-(4- | 31 |
| | (4-tert-butylbenzoylamino)- | tert-butylbenz- | |
| | phenyl]ethyl}-2-(3- | amido)-5- | |
| | trifluoromethyl- | iodobenzoate | |
| | benzoylamino)benzoic | | |
| | acid | | |
| 2:3 | 2-(4-Trifluoromethyl- | N-(4-Iodophenyl)- | 70 |
| | benzoylamino)-5-{2- | 3-(trifluoro- | |
| | [4-(3-trifluoromethyl- | methyl)benzamide | |
| | benzoylamino)-phenyl]- | | |
| | ethyl}benzoic acid | | |
| 2:4 | 5-{2-[3-Carboxy-4- | Methyl 5-iodo-2- | 70 |
| | (4-trifluoromethyl-benzoylamino)- | (3-trifluoro- | |
| | phenyl]ethyl}-2-(3- | methylbenzamido)- | |
| | trifluoromethyl- | benzoate | |
| | benzoylamino)benzoic | | |
| | acid | | |
| 2:5 | 2-(4-Trifluoromethyl- | N-(4-Iodophenyl)- | 57 |
| | benzoylamino)-5-[4- | 3-trifluoro- | |
| | (3-trifluoromethyl- | methylbenzamide | |
| | benzoylamino)-phenylethynyl]- | | |
| | benzoic acid | | |

### CH₂O Linker Compounds, Table 3

Step 1: A mixture of 2-amino-5-methylbenzoic acid (1.5 g, 10 mmol), 3-chlorobenzoyl chloride (3.7 g, 21 mmol) and pyridine (20 mL) was stirred at rt over night. The mixture was concentrated, water was added and the mixture stirred at it for 2 h. The mixture was filtered and the solids washed with water. The combined filtrates were concentrated and the residue dissolved in MeOH (30 mL). Sodium methoxide (0.27 g, 5 mmol) was added and the mixture stirred at it for 30 min. Concentration and addition of water gave a precipitate that was collected and washed with water. After drying, 2.7 g of methyl 2-(3-chlorobenzamido)-5-methylbenzoate was obtained.

Step 2: A mixture of methyl 2-(3-chlorobenzamido)-5-methylbenzoate (2.8 g, 9.0 mmol), N-bromosuccinimide (1.7 g, 9.4 mmol), benzoylperoxide (catalytic) and CCl₄ (60 mL) was stirred under UV-irradiation for 3 h at rx. The precipitate was collected and washed with CCl₄. Purification by chromatography and crystallization afforded methyl 5-(bromomethyl)-2-(3-chlorobenzamido)benzoate (820 mg).

Step 3: A mixture of methyl 2-(arylamido)-5-hydroxybenzoate (prepared according to Step 1 via aroylation of 2-amino-5-hydroxybenzoic acid) (150 mg, 0.5 mmol), methyl 5-(bromomethyl)-2-(3-chlorobenzamido)benzoate (191 mg, 0.5 mmol), K₂CO₃ (83 mg, 0.6 mmol), 18-crown-6-ether (catalytic) and DMF (3.5 mL) was stirred at it for 18 h. Dilution with water and stirring at 0° C. for 1 h gave a precipitate which was collected, washed with water and dried. Purification by chromatography gave esters that were hydrolyzed according to method B to furnish the title compounds in Table 3.

TABLE 3

| | | | Yield (%) | |
|---|---|---|---|---|
| | | | Ether | |
| No | Title Compound | Phenol in Step 3 | coupling | Hydrolysis |
| 3:1 | 2-(4-Butylbenzoyl- | Methyl 2- | 60 | 75 |
| | amino)-5-[3-carboxy- | (4-butylbenz- | | |
| | 4-(3-chlorobenzoyl- | amido)-5- | | |
| | amino)benzyloxy]- | hydroxy-benzoate | | |
| | benzoic acid | | | |
| 3:2 | 2-(3-Chlorobenzoyl- | Methyl 2- | 58 | 72 |
| | amino)-5-[3-carboxy- | (3-chloro- | | |
| | 4-(3-chlorobenzoyl- | benzamide)-5- | | |
| | amino)-benzyloxy]- | hydroxybenzoate | | |
| | benzoic acid | | | |

### Amide Linker Compounds, Table 4

### Synthesis of methyl 5-(4-aminobenzamido)-2-(4-butylbenzamido)benzoate

Step 1: A mixture of 4-butylbenzoyl chloride (1.0 mL, 5.6 mmol) and triethylamine (788 μL, 5.6 mmol) was added to a mixture of methyl 2-amino-5-nitrobenzoate (1.0 g, 5.1 mmol) and acetonitrile. The mixture was heated at rx for 1 d and allowed to cool. MeOH (20 mL) was added and stirring was continued for 20 min. After concentration, the residue was purified by recrystallisation from EtOH to give methyl 2-(4-butylbenzamido)-5-nitrobenzoate (1.61 g, 87%).

Step 2: A mixture of methyl 2-(4-butylbenzamido)-5-nitrobenzoate (1.61 g, 4.5 mmol), Pd/C (200 mg, 10%), EtOH (50 mL) and EtOAc (50 mL) was hydrogenated at ambient temperature and pressure for 1 h and filtered through Celite. The solids were washed with EtOAc and the combined filtrates concentrated. The residue was aroylated with 4-nitrobenzoyl chloride according to Step 1 to give methyl 2-(4-butylbenzamido)-5-(4-(4-nitrobenzamido)-benzamido)benzoate.

Step 3: The method in Step 2 was used with methyl 2-(4-butylbenzamido)-5-(4-(4-nitrobenzamido)benzamido)benzoate furnishing methyl 5-(4-aminobenzamido)-2-(4-butylbenzamido)benzoate in quantitative yield.

Step 4: The compound from Step 3 was benzoylated (Examples 4:1-2, table 4) according to method A or sulfonylated (example 4:3, Table 4) as follows: methyl 5-(4-aminobenzamido)-2-(4-butylbenzamido)benzoate (0.13 g, 0.29 mmol) in CH₂Cl₂ (30 mL) and THF (10 mL) was warmed to 60° C. and a mixture of 3-chloro-2-methyl-benzenesulfonyl chloride (0.072 g, 0.32 mmol), DMAP 0.7 mg, 0.0058 mmol) and triethylamine (44 μL, 0.32 mmol) was added. After heating at 60° C. over night, the mixture was cooled. Extractive workup (EtOAc, HCl (0.1 M, aq), brine), drying (Na₂SO₄) and purification by chromatography gave the methyl 2-(4-butylbenzamido)-5-(4-(3-chloro-N-(3-chloro-2-methylphenylsulfonyl)-2-methylphenylsulfonamido)benzamido)benzoate (65 mg, 27%).

Hydrolysis according to method B gave compounds 4:1-3 in Table 4.

TABLE 4

| | | | Yield (%) | |
|---|---|---|---|---|
| No | Title Compound | Reactant | Ester | Acid |
| 4:1 | 2-(4-Butylbenzoylamino)- | 4-Isopropoxybenzoyl | 80 | 94 |
| | 5-[4-(4-isopropoxy-benzoyl- | chloride | | |
| | amino)benzoyl-amino]- | | | |
| | benzoic acid | | | |
| 4:2 | 5-(4-Benzoylaminobenzoy- | Benzoyl chloride | 68 | 82 |
| | lamino)-2-(4-butyl- | | | |
| | benzoylamino)benzoic acid | | | |
| 4:3 | 2-(4-Butylbenzoylamino)- | 3-Chloro-2-methyl- | 27 | 94 |
| | 5-[4-(3-chloro-2-methyl- | benzenesulfonyl | | |
| | benzenesulfonylamino)- | chloride | | |
| | benzoylamino]-benzoic acid | | | |

### Preparation of asymmetric 2,5-diarylsulfonylamidobenzoic acids 5:1-5:5, Table 5

Step 1: NaH (306 mg, 12.75 mmol) was added in portions to methyl 2-amino-5-nitrobenzoate (1 g, 5.10 mmol) in THF. The mixture was stirred at rt for 15 min, The appropriate arylsulfonyl chloride (6.44 mmol) was added and the mixture was stirred at rt for 30 min. Water was added and the mixture was acidified with aq HCl. Extractive workup (EtOAc, water), drying (Na₂SO₄) and concentration furnished the corresponding methyl 2-arylsulfonylamino-5-nitrobenzoates.

Step 2: The compound from Step 1 (5 mmol) in EtOAc or MeOH was hydrogenated over 10% Pd/C (0.5 mmol) at ambient temperature and pressure. The mixture was filtered and concentrated to give the product in quantitative yield.

Step 3: A mixture of the compound from Step 1 (0.7 mmol), the appropriate arylsulfonyl chlorides (see Table 5) (0.77 mmol) and pyridine was stirred at rt overnight. After standard workup (EtOAc, dilute HU, water), drying (Na₂SO₄) and concentration the residue was hydrolyzed according to general method B to give the title compounds in yields given in Table 5.

TABLE 5

| | | | Yield |
|---|---|---|---|
| No | Title Compound | Reactant | (%) |
| 5:1 | 5-(4-Phenoxyphenylsulfonamido)- | 4-Phenoxyphenylsulfonyl | 11 |
| | 2-(4-(trifluoromethoxy)phenyl- | chloride | |
| | sulfonamido)benzoic acid | | |
| 5:2 | 2-(4-Butylphenylsulfonamido)-5- | 4-Phenoxyphenylsulfonyl | 98 |
| | (4-phenoxyphenylsulfonamido)- | chloride | |
| | benzoic acid | | |
| 5:3 | 2-(4-Butylphenylsulfonamido)-5- | 4-(2-Chlorophenoxy)- | 78 |
| | (4-(2-chloro-phenoxy)- | phenylsulfonyl chloride | |
| | phenylsulfonamido)benzoic acid | | |
| 5:4 | 2-(4-Butylphenylsulfonamido)-5- | 4-(2-Methoxyphenoxy)- | 95 |
| | (4-(2-methoxy-phenoxy) | phenylsulfonyl chloride | |
| | phenylsulfonamido)benzoic acid | | |
| 5:5 | 2-(4-Butoxyphenylsulfonamido)-5- | 4-(2- | 76 |
| | (4-(2-chloro-phenoxy)- | Chlorophenoxy)phenyl- | |
| | phenylsulfonamido)benzoic acid | sulfonyl chloride | |

### Preparation of 5-arylbenzylamino-2-(4-butouphenylsulfonamido)benzoic acids, Table 6

To a solution of methyl 5-amino-2-(4-butoxyphenylsulfonamido)benzoate (see Example 5:2, Step 1) (300 mg, 0.793 mmol) in MeOH was added arylaldehyde (1.6 mmol) followed by NaBH₄ (120 mg, 3.17 mmol). The mixture was stirred at rt overnight. Extractive workup (EtOAc, water), drying (Na₂SO₄), concentration and purification by chromatography followed by hydrolysis according to general method B gave the title compounds in yields given in Table 6.

TABLE 6

| | | | Yield |
|---|---|---|---|
| No | Title Compoundbe | Reactant | (%) |
| 6:1 | 2-(4-Butoxyphenylsulfonamido)- | 3-(4-tert-butylphenoxy)- | 97 |
| | 5-(3-(4-tert-butylphenoxy)benzyl- | benzaldehyde | |
| | amino)benzoic acid | | |
| 6:2 | 2-(4-Butoxyphenylsulfonamido)- | 3-(2-chlorobenzyloxy)- | 94 |
| | 5-(3-(2-chlorobenzyloxy)benzyl- | benzaldehyde | |
| | amino)benzoic acid | | |

TABLE 7
Spectroscopic data for the exemplified compounds in Tables 1-6

| No | ¹H NMR (DMSO-d₆, 400 or 300 or 200 MHz), δ: |
|---|---|
| 1:1 | 13.8-13.6 (2H, br s) 12.10 (2H, s) 8.63 (2H, d, J = 8.6 Hz) 8.04 (2H, d, J = 1.8 Hz) |
| | 7.89-7.78 (4H, m) 7.61 (2H, dd, J = 8.6, 1.8 Hz) 7.42-7.32 (4H, m) |
| | 6.11 (1H, d, J = 3.6 Hz) 5.78 (1H, d, J = 3.6 Hz) 2.64 (4H, t, J = 7.5 Hz) |
| | 1.65-1.46 (4H, m) 1.39-1.17 (4H, m) 0.87 (6H, t, J = 7.2 Hz) |
| 1:2 | 11.3 (br s, 2H), 8.10-8.00 (m, 2H) 7.90-7.77 (m, 6H), 7.68-7.47 (m, 8H); |
| 1:3 | 12.2 (s, 2H), 8.72 (d, J = 7.8 Hz, 2H), 8.29 (d, J = 6.6 Hz, 2H), 8.03 (dd, J = 7.8, |
| | 1.7 Hz, 2H), 7.99 (s, 2H), 7.93 (d, J = 7.2 Hz, 2H), 7.72 (d, J = 8.1 Hz, 2H), |
| | 7.63 (t, J = 7.8 Hz, 2H) |
| 1:4 | 13.64 (br s, 2H), 10.45 (s, 2H), 10.07 (s, 2H), 8.51-8.46 (m, 2H), |
| | 8.22-8.19 (m, 2H), 7.93-7.88 (m, 2H), 7.77-7.74 (m, 2H), 7.42-7.37 (m, 2H), |
| | 7.35-7.28 (m, 2H), 7.07-7.02 (m, 2H) |
| 1:5 | 11.32 (br s, 2H), 8.97 (d, J = 1.8 Hz, 2H), 8.80 (d, J = 3.6 Hz, 2H), 8.21 (d, |
| | J = 8.4 Hz, 2H), 8.05 (d, J = 2.1 Hz, 2H), 7.84 (dd, J = 8.4, 2.1 Hz, 2H), |
| | 7.61-7.56 (m, 2H) |
| 1:6 | 12.15 (s, 2H), 8.82 (d, J = 9.0 Hz, 2H), 8.30 (s, 2H), 7.90 (d, J = 9.0 Hz, 2H), |
| | 7.86 (d, J = 9.0 Hz, 4H), 7.60 (d, J = 8.0 Hz, 4H), 1.39 (s, 18H) |
| 1:7 | 11.09 (s, 2H), 8.55 (d, J = 9.0 Hz, 2H), 8.15 (s, 2H), 7.90 (d, J = 8.5 Hz, 2H), |
| | 7.42 (d, J = 7.8 Hz, 4H), 7.36 (d, J = 8.0 Hz, 4H), 3.80 (s, 4H) |
| 2:1 | 14.0-13.4 (2H, br s) 12.22 (1H, s) 12.04 (1H, s) 8.62 (1H, d, J = 8.6 Hz) |
| | 8.54 (1H, d, J = 8.6 Hz) 8.30-8.19 (2H, m) 8.10-7.76 (6H, m) 7.63-7.46 (2H, m) |
| | 7.20-7.04 (2H, m) 3.85 (3H, s) 2.93 (4H, s) |
| 2:2 | 13.9-13.6 (2H, br s) 12.19 (1H, s) 12.07 (1H, s) 8.64 (1H, d, J = 8.5 Hz) |
| | 8.54 (1H, d, J = 8.4 Hz) 8.31-8.20 (2H, m) 8.08-7.79 (6H, m) 7.67-7.50 (4H, m) |
| | 2.94 (4H, s) 1.36 (9H, s) |
| 2:3 | 14.0-13.6 (1H, br s) 12.15 (1H, s) 10.41 (1H, s) 8.55 (1H, d, J = 8.6 Hz) |
| | 8.30-8.08 (4H, m) 8.05-7.89 (4H, m) 7.85-7.62 (3H, m) 7.54 (1H, dd, J = 8.6, 2.0 Hz) |
| | 7.32-7.17 (2H, m) 2.96-2.86 (4H, m) |
| 2:4 | 14.0-13.6 (2H, br s) 12.20 (1H, s) 12.18 (1H, s) 8.56 (1H, d, J = 8.5 Hz) |
| | 8.55 (1H, d, J = 8.6 Hz) 8.36-7.90 (10H, m) 7.59 (1H, d, J = 8.5 Hz) 7.57 (1H, d, |
| | J = 8.6 Hz) 2.95 (4H, s) |
| 2:5 | 12.5-12.4 (1H, br s) 10.67 (1H, s) 8.74 (1H, d, J = 8.8 Hz) 8.34-8.24 (2H, m) |
| | 8.22-8.11 (3H, m) 8.05-7.96 (3H, m) 7.93-7.75 (4H, m) 7.66-7.56 (2H, m) |
| 3:1 | 12.18 (1H, s) 11.83 (1H, s) 8.69-8.56 (2H, m) 8.15 (1H, d, J = 2.0 Hz) |
| | 7.98-7.59 (8H, m) 7.43-7.34 (3H, m) 5.19 (2H, s) 2.67 (2H, t, J = 7.3 Hz) |
| | 1.68-1.49 (2H, m) 0.90 (3H, t, J = 7.3 Hz) |
| 3:2 | 12.25 (1H, s) 11.88 (1H, s) 8.65 (1H, d, J = 8.8 Hz) 8.49 (1H, d, J = 8.8 Hz) |
| | 8.16 (1H, d, J = 2.0 Hz) 8.00-7.85 (4H, m) 7.81-7.57 (6H, m) 7.38 (1H, dd, |
| | J = 8.8, 2.9 Hz) 5.21 (2H, s) |
| 4:1 | 12.00 (1H, s) 10.32 (1H, s) 10.30 (1H, s) 8.67 (1H, d, J = 9.2 Hz) 8.55 (1H, d, |
| | J = 2.6 Hz) 8.10-7.81 (9H, m) 7.45-7.34 (2H, m) 7.09-6.97 (2H, m) 4.74 (1H, |
| | septet, J = 5.9 Hz) 2.73-2.59 (2H, m) 1.68-1.49 (2H, m) 1.40-1.19 (2H, m) |
| | 1.29 (6H, d, J = 5.9 Hz) 0.89 (3H, t, J = 7.1 Hz) |
| 4:2 | 12.01 (1H, s) 10.52 (1H, s) 10.32 (1H, s) 8.67 (1H, d, J = 9.2 Hz) 8.55 (1H, d, |
| | J = 2.6 Hz) 8.10-7.79 (9H, m) 7.67-7.48 (3H, m) 7.45-7.33 (2H, m) |
| | 2.73-2.58 (2H, m) 1.68-1.48 (2H, m) 1.42-1.18 (2H, m) 0.89 (3H, t, J = 7.1 Hz) |
| 4:3 | 12.01 (1H, s) 11.08 (1H, s) 10.23 (1H, s) 8.63 (1H, d, J = 9.3 Hz) 8.47 (1H, d, |
| | J = 2.0 Hz) 8.12-7.91 (2H, m) 7.90-7.77 (4H, m) 7.73 (1H, d, J = 9.3 Hz) |
| | 7.48-7.33 (3H, m) 7.22-7.10 (2H, m) 2.72-2.58 (2H, m) 2.64 (3H, s) |
| | 1.66-1.48 (2H, m) 1.40-1.17 (2H, m) 0.88 (3H, t, J = 7.0 Hz) |
| 5:1 | 10.87 (br s, 1H), 10.25 (s, 1H), 7.88-7.83 (m, 2H), 7.67-7.62 (m, 2H), |
| | 7.57-7.54 (m, 1H), 7.51-7.37 (m, 5H), 7.33-7.22 (s, 2H), 7.11-7.00 (m, 4H) 28516 |
| 5:2 | 10.73 (br s, 1H), 10.19 (s, 1H), 7.64-7.58 (m, 4H), 7.55-7.52 (m, 1H), |
| | 7.48-7.41 (m, 3H), 7.32-7.23 (m, 4H), 7.10-6.99 (m, 4H), 2.56 (t, J = 7.6 Hz, 2H), |
| | 1.53-1.43 (m, 2H), 1.28-1.17 (m, 2H), 0.84 (t, J = 7.3 Hz, 3H) |
| 5:3 | 10.75 (br s, 1H), 10.19 (s, 1H), 7.67-7.58 (m, 5H), 7.55-7.52 (m, 1H), |
| | 7.46-7.40 (m, 2H), 7.36-7.22 (m, 5H), 6.99-6.93 (m, 2H), 2.57 (t, J = 7.6 Hz, 2H), |
| | 1.54-1.44 (m, 2H), 1.28-1.17 (m, 2H), 0.84 (t, J = 7.3 Hz, 3H) |
| 5:4 | 10.72 (br s, 1H), 10.16 (s, 1H), 7.64-7.52 (m, 5H), 7.42-7.39 (m, 1H), |
| | 7.31-7.21 (m, 4H), 7.21-7.18 (m, 1H), 7.14-7.08 (m, 1H), 7.04-6.98 (m, 1H), |
| | 6.88-6.83 (m, 2H), 3.65 (s, 3H), 2.57 (t, J = 7.6 Hz, 2H), 1.54-1.44 (m, 2H), |
| | 1.28-1.17 (m, 2H), 0.84 (t, J = 7.3 Hz, 3H) |
| 5:5 | 10.67 (br s, 1H), 10.18 (s, 1H), 7.66-7.59 (m, 5H), 7.54-7.51 (m, 1H), |
| | 7.46-7.41 (m, 2H), 7.36-7.21 (m, 3H), 7.01-6.98 (m, 4H), 3.97 (t, J = 6.5 Hz, 2H), |
| | 1.69-1.61 (m, 2H), 1.43-1.32 (m, 2H), 0.88 (t, J = 7.4 Hz, 3H) |
| 5:6 | 10.66 (br s, 1H), 10.25 (s, 1H), 7.71-7.62 (m, 3H), 7.60-7.53 (m, 3H), |
| | 7.47-7.23 (m, 7H), 6.99-6.92 (m, 2H), 2.57 (t, J = 7.6 Hz, 2H), 1.55-1.43 (m, 2H), |
| | 1.28-1.16 (m, 2H), 0.84 (t, J = 7.3 Hz, 3H) |
| 5:7 | 10.71 (br s, 1H), 10.21 (s, 1H), 7.62-7.58 (m, 2H), 7.57-7.52 (m, 3H), |
| | 7.42-7.38 (m, 1H), 7.34-7.30 (m, 4H), 7.29-7.25 (m, 1H), 2.63-2.57 (m, 4H), |
| | 1.55-1.46 (m, 4H), 1.30-1.20 (m, 4H), 0.85 (t, J = 7.4 Hz, 6H) |
| 6:1 | 10.07 (s, 1H), 7.54-7.48 (m, 2H), 7.35-7.23 (m, 4H), 7.08-7.04 (m, 1H), |
| | 7.02-6.99 (m, 1H), 6.98-6.92 (m, 3H), 6.86-6.79 (m, 3H) 6.77-6.73 (m, 1H), |
| | 4.21 (s, 2H), 3.96 (t, J = 6.5 Hz, 2H), 1.69-1.61 (m, 2H), 1.44-1.33 (m, 2H), |
| | 1.26 (s, 9H), 0.90 (t, J = 7.4 Hz, 3H) |
| 6:2 | 10.04 (s, 1H), 7.57-7.47 (m, 4H), 7.40-7.32 (m, 2H), 7.27-7.21 (m, 2H), |
| | 7.05-7.02 (m, 1H), 7.00-6.95 (m, 3H), 6.94-6.90 (m, 1H) 6.89-6.86 (m, 1H), |
| | 6.81-6.76 (m, 1H), 5.11 (s, 2H), 4.19 (s, 2H), 3.97 (t, J = 6.5 Hz, 2H), |
| | 1.70-1.62 (m, 2H), 1.44-1.34 (m, 2H), 0.90 (t, J = 7.4 Hz, 3H) |

### Example 7

Title compounds of the examples were tested in the biological test described above (in vitro assay) and were found to exhibit the following percentage inhibitions of LTC₄ at a concentration of 10 μM. For example, the following representative compounds of the examples exhibited the percentage inhibitions:

| Ex. | Percentage Inhibition at 10 μM |
|---|---|
| 1:2 | 92 |
| 1:3 | 88 |
| 1:4 | 99 |
| 1:5 | 69 |
| 1:6 | 100 |
| 1:7 | 100 |
| 1:8 | 100 |
| 1:9 | 89 |
| 1:10 | 98 |
| 1:11 | 95 |
| 1:12 | 97 |
| 2:1 | 87 |
| 2:2 | 100 |
| 2:3 | 85 |
| 2:4 | 98 |
| 2:5 | 89 |
| 3:1 | 100 |
| 3:2 | 100 |
| 4:1 | 79 |
| 4:2 | 96 |
| 4:3 | 100 |
| 5:1 | 100 |
| 5:2 | 100 |
| 5:3 | 98 |
| 5:4 | 100 |
| 5:5 | 99 |
| 6:1 | 100 |
| 6:2 | 100 |

1. A compound of formula I, 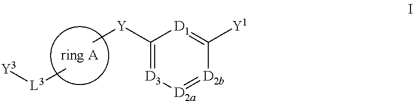
wherein
either one of D₂ₐ and D_{2b} represents D₂, and the other represents —C(-L²-Y²)═;
Y represents a direct bond, —C(R^{b1})(R^{b2})—C(R^{b3})(R^{b4})—, —C(R^{c1})═C(R^{c2})—, —C≡C—, —O—C(R^{d1})(R^{d2})—, —C(R^{e1})(R^{e2})—O—, —N(R^{f1})—C(O)—, —C(O)—N(R^{g1})—, —C(OR^{q1})(R^{h1})—, —N(Rⁱ¹)—C(R^{j1})(R^{j2})—, —C(R^{k1})(R^{k2})—N(R^{m1})—, —N(Rⁿ¹)—S(O)₂— or —S(O)₂—N(R^{p1})—;
R^{b1}, R^{b2}, R^{b3}, R^{b4}, R^{c1}, R^{c2}, R^{d1}, R^{d2}, R^{e1}, R^{e2}, R^{f1}, R^{g1}, R^{h1}, Rⁱ¹, R^{j1}, R^{j2}, R^{k1}, R^{k2}, R^{m1}, Rⁿ¹, R^{p1} and R^{q1} independently represent hydrogen or C₁₋₆ alkyl optionally substituted by one or more substituents selected from ═O, halo and —OR^{s1}; or
Rⁱ¹, R^{m1} and R^{q1} may independently represent —S(O)₂R^{r1}; or
any two of R^{b1}, R^{b2}, R^{b3}, R^{b4}, R^{c1}, R^{c2}, R^{d1}, R^{d2}, R^{e1}, R^{e2}, R^{j1}, R^{j2}, R^{k1} and R^{k2} when attached to the same or adjacent carbons atoms (i.e. the following combinations: R^{b1} and R^{b2}; R^{b3} and R^{b4}; either one of R^{b1} and R^{b2} and either one of R^{b3} and R^{b4}; R^{c1} and R^{c2}; R^{d1} and R^{d2}; R^{e1} and R^{e2}; R^{j1} and R^{j2}; or R^{k1} and R^{k2}) may be linked together to form, together with the carbon atom(s) to which they are attached, a 3- to 8-membered ring optionally containing one to three unsaturations, one to three heteroatoms, and which ring is optionally substituted by one or more substituents selected from halo and C₁₋₃ alkyl (optionally substituted by one or more substituents selected from ═O and halo);
R^{r1} represents C₁₋₆ alkyl optionally substituted by one or more fluoro atoms;
R^{s1} represents hydrogen or C₁₋₆ alkyl optionally substituted by one or more fluoro atoms;
each of D₁, D₂ and D₃ respectively represent —C(R^{1a})═, —C(R^{1b})═ and —C(R^{1c})═;
ring A represents: 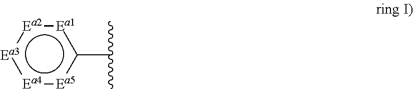
each of E^{a1}, E^{a2}, E^{a3}, E^{a4} and E^{a5} respectively represent —C(R^{2a})═, —C(R^{2b})═, —C(R^{2e})═, —C(R^{2d})═ and —C(R^{2a})═, or, each of E^{a1}, E^{a2}, E^{a3}, E^{a4} and E^{a5} may alternatively and independently represent —N═;
R^{2a} and R^{2e} independently represent hydrogen, —Y^{1a} or a substituent selected from X¹;
one of R^{2b}, R^{2c} and R^{2d} represents the requisite -L³-Y³ group, and the others independently represent hydrogen, —Y^{1a} or a substituent selected from X¹; 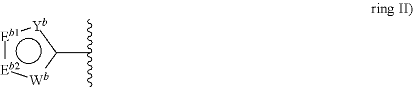
E^{b1} and E^{b2} respectively represent —C(R^{3a})═ and —C(R^{3b})═;
Y^{b} represents —C(R^{3c})═ or —N═;
W^{b} represents —N(R^{3d})—, —O— or —S—;
one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, —Y^{1a} or a substituent selected from X², and the remaining R^{3d} substituent (if present) represents hydrogen or a substituent selected from R^{z1}; or 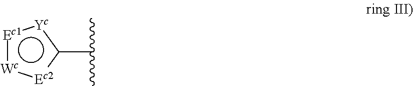
E^{c1} and E^{c2} each respectively represent —C(R^{4a})═ and —C(R^{4b})═;
Y^{c} represents —C(R^{4c})═ or —N═;
W^{c} represents —N(R^{4d})—, —O— or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, —Y^{1a} or a substituent selected from X³, and the remaining R^{4d} substituent (if present) represents hydrogen or a substituent selected from R^{z2};
R^{z1} and R^{z2} independently represent a group selected from Z^{1a};
R^{1a}, R^{1b} and R^{1c} independently represent hydrogen, a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, or, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent, —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or
any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O, —OR^{5h} and R^{5a};
R⁵ⁱ, R^{5m} and R^{5p} independently represent R^{5a};
R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and —S(O)₂-M¹;
R^{8C}, R^{8f} and R^{8h} independently represent hydrogen, —S(O)₂CH₃, —S(O)₂CF₃ or C₁₋₆ alkyl optionally substituted by one or more substituents selected from F, Cl, ═O, —OR^{13a}, —N(R^{14a})R^{14b} and —S(O)₂-M²; or
R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O or C₁₋₃ alkyl optionally substituted by one or more substituents selected from ═O and fluoro;
M¹ and M² independently represent —N(R^{15a})R^{15b} or C₁₋₃ alkyl optionally substituted by one or more fluoro atoms;
R^{11a} and R^{13a} independently represent H or C₁₋₃ alkyl optionally substituted by one or more fluoro atoms;
R^{12a}, R^{12b}, R^{14a}, R^{14b}, R^{15a} and R^{15b} independently represent hydrogen, —CH₃ or —CH₂CH₃;
Y¹ and Y^{1a} independently represent, on each occasion when used herein, —C(O)OR^{9a};
R^{9a} represents, on each occasion when used herein, hydrogen, C₁₋₈ alkyl, a heterocycloalkyl group (which latter two groups are optionally substituted by one or more substituents selected from G¹ and/or Z¹), an aryl group or a heteroaryl group (which latter two groups are optionally substituted by one or more substituents selected from G¹);
Y² and Y³ independently represent an aryl group or a heteroaryl group, both of which groups are optionally substituted by one or more substituents selected from A;
A represents, on each occasion when used herein:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when used herein, halo, cyano, —N₃, —NO₂, —ONO₂ or -A¹-R^{16a};
wherein A¹ represents a single bond or a spacer group selected from —C(O)A²-, —S—, —S(O)₂A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;
Z¹ represents, on each occasion when used herein, ═O, ═S, ═NOR^{16b}, ═NS(O)₂N(R^{17f})R^{16c}, ═NCN or ═C(H)NO₂;
B represents, on each occasion when used herein:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when used herein, halo, cyano, —N₃, —NO₂, —ONO₂ or -A⁶-R^{18a};
wherein A⁶ represents a single bond or a spacer group selected from —C(O)A⁷-, —S—, —S(O)₂A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂ or —S(O)₂N(R^{19e})—;
Z² represents, on each occasion when used herein, ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when used herein, halo, cyano, —N₃, —NO₂, —ONO₂ or -A¹¹-R^{20a};
wherein A¹¹ represents a single bond or a spacer group selected from —C(O)A¹²-, —S—, —S(O)₂A¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;
Z³ represents, on each occasion when used herein, ═O, ═S, ═NOR^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L² and L³ independently represent a spacer group selected from —N(R^{24a})-A¹⁶-, and —OA¹⁷-;
A¹⁶ represents a direct bond, —C(O)—, —C(O)N(R^{25a})—, —C(O)CH₂— or —S(O)₂—;
A¹⁷ represents a direct bond or —CH₂—;
m represents, on each occasion when used herein, 0, 1 or 2;
R^{22a}, R^{22b}, R^{22c}, R^{22d}, R^{22e}, R^{22f}, R^{23a}, R^{23b}, R^{23c}, R^{24a} and R^{25a} are independently selected from hydrogen and C₁₋₄ alkyl, which latter group is optionally substituted by one or more substituents selected from fluoro, —OH, —OCH₃, —OCH₂CH₃ and/or ═O,
or a pharmaceutically-acceptable salt thereof,
provided that when:
(A) D₂ₐ represents D₂ and D_{2b} represents —C(-L²-Y²)═; ring A represents ring I); E^{a1}, E^{a2}, E^{a3}, E^{a4} and E^{a5} respectively represent —C(R^{2a})═, —C(R^{2b})═, —C(R^{2b})═, —C(R^{2d})═ and —C(R^{2e})═; R^{1a}, R^{1b}, R^{1c}, R^{2a}, R^{2d} and R^{2e} all represent hydrogen; Y¹ and Y^{1a} both represent —C(O)OR^{9a}; R^{9a} represents hydrogen:
(I) Y represents a direct bond; R^{2b} represents —Y^{1a}; R^{2c} represents -L³-Y³:
(a) L² and L³ both represent —N(H)C(O)—, then Y² and Y³ do not both represent 4-fluorophenyl;
(b) L² and L³ both represent —N(H)C(O)—, then Y² and Y³ do not both represent: unsubstituted phenyl; unsubstituted 1-naphthyl; 2-fluorophenyl; 3-fluorophenyl; 2-chlorophenyl; 4-chlorophenyl; 3-bromophenyl; 4-bromophenyl; 2-iodophenyl; 4-iodophenyl; 2-aminophenyl; 2-nitrophenyl; 3-nitrophenyl; 2-methylphenyl; 4-tert-butylphenyl; 2-acetoxyphenyl; 4-acetoxyphenyl; 3-acetamidophenyl; 4-acetamidophenyl; 3-(2,2-dimethyl-1-oxopropylamino)phenyl (i.e. 3-(2,2-dimethyl-propionamido)phenyl); 4-(2,2-dimethyl-1-oxopropyl-amino)phenyl (i.e. 4-(2,2-dimethyl-propionamido)phenyl); 4-biphenyl; 4-acetoxyophenyl; 4-methoxyphenyl; 3-ethoxyphenyl; 4-ethoxyphenyl, 4-n-propyloxyphenyl, 3,5-dimethyoxyphenyl; 2-methyl-3-nitrophenyl; 4-methyl-3-nitrophenyl; 4-(4-nitrophenoxy)phenyl; 3,4-dichlorophenyl; 2-chloro-4-nitrophenyl; 2-(carboxy)phenyl; 2-carboxy-4-nitrophenyl; 1,3-dihydro-1,3-dioxo-5-isobenzofuranyl or 4-[(1,3-dihydro-1,3-dioxo-5-isobenzofuranyl)oxy]phenyl;
(c) L² and L³ represent —N(H)—S(O)₂—, then Y² and Y³ do not both represent 4-methylphenyl;
(d) L² represents —N(H)—C(O)—; Y² represents 2,4-dichlorophenyl; then L³ does not represent —O— or —O—CH₂— when Y³ represents unsubstituted phenyl;
(e) L² and L³ represent —N(H)—, then Y² and Y³ do not both represent 5-hydroxy-7-(sulfonic acid)naphth-2-yl, 8-hydroxy-6-(sulfonic acid)naphth-2-yl or 2-anthraquinone;
(II) Y represents a direct bond; R^{2c} represents —Y^{1a}; R^{2b} represents -L³-Y³; L² and L³ both represent —N(H)C(O)—, then Y² and Y³ do not both represent 4-biphenyl;
(B) ring A represents ring I); E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═; R^{9a} represents hydrogen:
(I) Y represents —N(H)—C(O)—; D_{2b} represents D₂; D₂ₐ represents —C(-L²-Y²)═; R^{1a}, R^{1b}, R^{1c}, R^{2a}, R^{2b}, R^{2d} and R^{2e} all represent hydrogen; R^{2c} represents -L³-Y³; L² and L³ represent —N(H)C(O)—, then Y² and Y³ do not both represent 2-carboxy-4-nitrophenyl or 4-amino-2-carboxy-phenyl;
(II) Y represents —CH₂—CH₂—; D₂ₐ represents D₂; D_{2b} represents —C(-L²-Y²)═; R^{1a}, R^{1b}, R^{1c}, R^{2a}, R^{2d} and R^{2e} all represent hydrogen; R^{2b} represents Y^{1a}; R^{2c} represents -L³-Y³; L² and L³ represent —N(H)C(O)—; Y¹ and Y^{1a} both represent —C(O)OR^{9a}, then Y² and Y³ do not both represent 3,5-dimethoxyphenyl;
(C) Y represents a direct bond; D_{2b} represents D₂ and D₂ₐ represents —C(-L²-Y²)═; ring A represents ring I); E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═; R^{1c} represents chloro; R^{1a}, R^{1b}, R^{2a}, R^{2d} and R^{2e} all represent hydrogen; R^{2c} represents —Y^{1a}; R^{2b} represents -L³-Y³; Y¹ and Y^{1a} both represent —C(O)OR^{9a}; R^{9a} represents hydrogen; L² and L³ both represent —N(H)—, then Y² and Y³ do not both represent 2-carboxyphenyl.

2. A compound as claimed in claim 1, wherein Y represents a direct bond,
—C(R^{b1})(R^{b2})—C(R^{b3})(R^{b4})—, —C≡C—, —O—C(R^{d1})(R^{d2})—, —C(R^{e1})(R^{e2})—O—, —N(R^{f1})—C(O)—, —C(O)—N(R^{g1})—, —C(OH)(R^{h1})—, —N(Rⁱ¹)—C(R^{j1})(R^{j2})—, —C(R^{k1})(R^{k2})—N(R^{m1})—, —N(Rⁿ¹)—S(O)₂— or —S(O)₂—N(R^{p1})—.

3. A compound as claimed in claim 2, wherein Y represents —O—C(R^{d1})(R^{d2})—, —C(O)—N(R^{g1})—, —N(Rⁱ¹)—C(R^{j1})(R^{j2})—, —S(O)₂—N(R^{p1})— or, more preferably, —C(R^{b1})(R^{b2})—C(R^{b3})(R^{b4})—, —C(R^{e1})(R^{e2})—O—, —C≡C—, —N(R^{f1})—C(O)—, —C(OH)(R^{h1})—, —C(R^{k1})(R^{k2})—N(R^{m1})— or —N(Rⁿ¹)—S(O)₂—.

4. A compound as claimed in claim 1, wherein ring A represents ring (I).

5. A compound as claimed in claim 1, wherein E^{a1} and E^{a5} independently represent —C(H)═ and E^{a2}, E^{a3} and E^{a4} respectively represent —C(R^{2b})═, —C(R^{2c})═ and —C(R^{2d})═.

6. A compound as claimed in claim 1, wherein one of R^{2b} or R^{2c} represents the requisite -L³-Y³ group and the other represents hydrogen or —Y^{1a}; and/or wherein R^{2d} represents hydrogen.

7. A compound as claimed in claim 1, wherein R^{b1}, R^{b2}, R^{b3}, R^{b4}, R^{c1}, R^{c2}, R^{d1}, R^{d2}, R^{e1}, R^{e2}, R^{f1}, R^{g1}, R^{h1}, Rⁱ¹, R^{j1}, R^{j2}, R^{k1}, R^{k2}, R^{m1}, Rⁿ¹ and R^{p1} independently represent C₁₋₄ alkyl (optionally substituted by one or more fluoro atoms) or hydrogen.

8. A compound as claimed in claim 1, wherein Y represents a direct bond, —C(O)N(H)—, —S(O)₂N(H)—, —OCH₂—, —N(H)—CH₂—,
—C(OH)(H)—, —CH₂CH₂—, —C≡C—, —CH₂—O—, —N(H)C(O)—, —N(H)S(O)₂— or —CH₂—N(H)—.

9. A compound as claimed in claim 1, wherein R^{9a} represents C₁₋₆ alkyl or H.

10. A compound as claimed in claim 1, wherein A represents I) C₁₋₈ alkyl optionally substituted by one or more substituents selected from G¹; or II) G¹.

11. A compound as claimed in claim 1, wherein G¹ represents halo, cyano, —NO₂ or -A¹-R^{16a}.

12. A compound as claimed in claim 1, wherein A¹ represents a single bond, —C(O)A²-, —S—, —S(O)₂A³-, —N(R^{17a})A⁴- or —OA⁵-.

13. A compound as claimed in claim 1, wherein A¹⁶ represents a —C(O)—, —C(O)N(R^{25a})—, —C(O)CH₂— or —S(O)₂—.

14. A compound as claimed in claim 1, wherein L² and L³ represent —N(H)C(O)—, —N(H)C(O)N(H)—, —N(H)S(O)₂—, —O— or —OCH₂—.

15. A compound as claimed in claim 1, wherein R^{16a} represents hydrogen or C₁₋₈ alkyl (optionally substituted by one or more groups selected from G³.

16. A compound as claimed in claim 1, wherein G³ represents halo.

17. A compound as claimed in claim 1, wherein Y² and Y³ independently represent optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group.

18. A compound as claimed in claim 17, wherein Y² and Y³ independently represent optionally substituted naphthyl, phenyl or pyridyl.

19. A compound as claimed in claim 17 or claim 18, wherein the optional substituents are selected from halo; cyano; —NO₂; C₁₋₆ alkyl optionally substituted with one or more halo groups; heterocycloalkyl optionally substituted by one or more substituents selected from C₁₋₃ alkyl and ═O; —OR²⁶; —SR²⁶; —C(O)R²⁶; —C(O)OR²⁶; —N(R²⁶)R²⁷; and —S(O)₂R²⁸; wherein R²⁶ and R²⁷ independently represent H, C₁₋₆ alkyl optionally substituted by one or more halo groups or aryl optionally substituted by one or more halo or C₁₋₃ alkyl groups (which alkyl group is optionally substituted by one or more halo atoms); and R²⁸ represents aryl or C₁₋₆ alkyl.

20. A compound as defined in claim 1 but without all of the provisos except (A)(I)(a), (A)(I)(d) and (B)(II), or a pharmaceutically-acceptable salt thereof, for use as a pharmaceutical.

21. A pharmaceutical formulation including a compound as defined in claim 1 but without all of the provisos except (A)(I)(a), (A)(I)(d) and (B)(II), or a pharmaceutically-acceptable salt thereof, in admixture with a pharmaceutically acceptable adjuvant, diluent or carrier.

22. A compound as defined in claim 1 but without the provisos, or a pharmaceutically acceptable salt thereof, for use in the treatment of a disease in which inhibition of the synthesis of leukotriene C₄ is desired and/or required.

23. Use of a compound of formula I, as defined in claim 1 but without the provisos, or a pharmaceutically acceptable salt thereof, for the manufacture of a medicament for the treatment of a disease in which inhibition of the synthesis of leukotriene C₄ is desired and/or required.

24. A compound as claimed in claim 22, or a use as claimed in claim 23, wherein the disease is a respiratory disease, inflammation and/or has an inflammatory component.

25. A compound or use as claimed in claim 24 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anemia, hypereosinofilic syndrome, or a malignancy.

26. A compound or use as claimed in claim 25, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

27. A method of treatment of a disease in which inhibition of the synthesis of leukotriene C₄ is desired and/or required, which method comprises administration of a therapeutically effective amount of a compound of formula I as defined in claim 1 but without the provisos, or a pharmaceutically-acceptable salt thereof, to a patient suffering from, or susceptible to, such a condition.

28. A combination product comprising:
(A) a compound of formula I as defined in claim 1 but without the provisos, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

29. A combination product as claimed in claim 28 which comprises a pharmaceutical formulation including a compound of formula I as defined in claim 1 but without the provisos, or a pharmaceutically-acceptable salt thereof, another therapeutic agent that is useful in the treatment of a respiratory disorder and/or inflammation, and a pharmaceutically-acceptable adjuvant, diluent or carrier.

30. A combination product as claimed in claim 28 which comprises a kit of parts comprising components:
(a) a pharmaceutical formulation including a compound of formula I as defined in claim 1 but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
which components (a) and (b) are each provided in a form that is suitable for administration in conjunction with the other.

31. A process for the preparation of a compound of formula I as defined in claim 1, which process comprises:
(i) for compounds of formula I in which L² and/or L³ represents —N(R^{24a})A¹⁶- in which R^{24a} represents H, reaction of a compound of formula II, 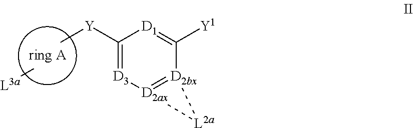
or a protected derivative thereof wherein one of D₂ₐₓ and D_{2bx} represents D₂ and the other represents —C(-L^{2a})= (i.e. the L^{2a} substituent is attached to either one of D₂ₐₓ and D_{2bx}), L^{2a} represents —NH₂ or -L²-Y², L^{3a} represents —NH₂ or -L³-Y³, provided that at least one of L^{2a} and L^{3a} represents —NH₂, and ring A, D₁, D₂, D₃, Y and Y¹ are as defined in claim 1, with:
(A) when A¹⁶ represents —C(O)N(R^{25a})—, in which R^{25a} represents H:
(a) a compound of formula III,
Y^{a}—N═C═O; III
or
(b) with CO (or a reagent that is a suitable source of CO ( ) or a reagent such as phosgene or triphosgene in the presence of a compound of formula IV,
Y^{a}—NH₂ IV
wherein, in both cases, Y^{a} represents Y² or Y³ (as appropriate/required) as defined in claim 1;
(B) when A¹⁶ represents a direct bond, with a compound of formula VI,
Y^{a}-L^{a} VI
wherein L^{a} represents a suitable leaving group;
(C) when A¹⁶ represents —S(O)₂—, —C(O)— or —C(O)CH₂—, with a compound of formula VII,
Y^{a}-A^{16a}-L^{a} VII
wherein A^{16a} represents —S(O)₂—, —C(O)— or —C(O)CH₂—, and Y^{a} and L^{a} are as defined in claim 1;
(ii) for compounds of formula I in which one of L² and L³ represents —N(R^{24a})C(O)N(R^{25a})— (and the other represents —NH₂ (or a protected derivative thereof), —N(R^{24a})C(O)N(R^{25a})— or another L² or L³ group as defined herein), in which R^{24a} and R^{25a} both represent H, reaction of a compound of formula VIII, 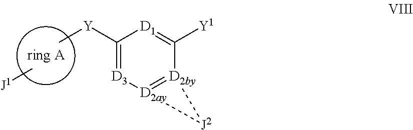
wherein one of D_{2ay} and D_{2by} represents D₂ and the other represents —C(-J²)= (i.e. the J² substituent is attached to either one of D₂ₐₓ and D_{2bx}), one of J¹ or J² represents —N═C═O and the other represents -L²-Y² or -L³-Y³ (as appropriate), —NH₂ (or a protected derivative thereof) or —N═C═O (as appropriate), and ring A, D₁, D₂, D₃, Y and Y¹ are as defined in claim 1, with a compound of formula IV as defined above;
(iii) reaction of a compound of formula IX, 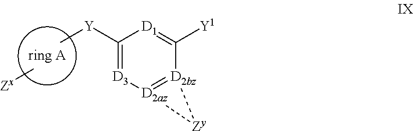
wherein one of D_{2az} and D_{2bz} represents D₂ and the other represents —C(—Z^{y})═ (i.e. the Z^{y} substituent is attached to either one of D_{2az} and D_{2bz}), one of Z^{x} and Z^{y} represents a suitable leaving group and the other represents -L²-Y² or -L³-Y³ (as appropriate) or the other may also represent a suitable leaving group, and ring A, D₁, D₂, D₃, Y and Y¹ are as defined in claim 1, with a (or two separate) compound(s) (as appropriate/required) of formula X,
Y^{a}-L^{x}-H X
wherein L^{x} represents L² or L³ (as appropriate/required), and Y^{a} is as defined above;
(iv) compounds of formula I in which there is a R^{f1}, R^{g1}, Rⁱ¹, R^{m1}, Rⁿ¹, R^{p1}, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ or R²⁵ group present, which is attached to a heteroatom such as nitrogen or oxygen, and which does/do not represent hydrogen), may be prepared by reaction of a corresponding compound of formula I in which such a group is present that does represent hydrogen with a compound of formula XI,
R^{wy}-L^{b} XI
wherein R^{wy} represents a R^{f1}, R^{g1}, Rⁱ¹, R^{m1}, Rⁿ¹, R^{p1} or R⁵ to R²⁵ group in which those groups do not represent hydrogen, and L^{b} represents a suitable leaving group;
(v) compounds of formula I in which there is a R^{f1}, R^{g1}, Rⁱ¹, R^{m1}, Rⁿ¹, R^{p1} or R⁵ to R²⁵ group present, which is attached to a heteroatom such as nitrogen or oxygen, and which does/do not represent hydrogen, an aryl group or a hetereoaryl group, may be prepared by reaction of a corresponding compound of formula I in which such a group is present that does represent hydrogen with a compound of formula XII,
R^{wy}-L^{c} XII
wherein R^{wy} represents any R^{wy} group as defined above (as appropriate; and preferably represents an aryl or heteroaryl group (if applicable), for the conversion to the appropriate compound of formula I) as defined in claim 1, and L^{c} represents a suitable leaving group;
(vi) for compounds of formula I that contain saturated alkyl groups, reduction of a corresponding compound of formula I that contains an unsaturation;
(vii) for compounds of formula I in which R^{9a} represents hydrogen, hydrolysis of a corresponding compound of formula I in which R^{9a} does not represent H;
(viii) for compounds of formula I in which Y¹ and/or, if present, Y^{1a} represents —C(O)OR^{9a}, and R^{9a} does not represent H:
(A) esterification (or the like) of a corresponding compound of formula I in which R^{9a} represents H; or
(B) trans-esterification (or the like) of a corresponding compound of formula I in which R^{9a} does not represent H (and does not represent the same value of the corresponding R^{9a} group in the compound of formula Ito be prepared),
in the presence of the appropriate alcohol of formula XIII,
R^{9za}OH XIII
in which R^{9za} represents R^{9a} provided that it does not represent H;
(ix) for compounds of formula I in which Y¹ and/or, if present, Y^{1a} represents —C(O)OR^{9a}, in which R^{9a} is other than H, reaction of a compound of formula XIV, 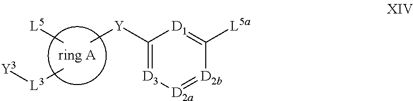
wherein at least one of L⁵ and L^{5a} represents an appropriate alkali metal group, a —Mg-halide, a zinc-based group or a suitable leaving group, and ring A, D₁, D₂ₐ, D_{2b}, D₃, Y, L³ and Y³ are as defined in claim 1, with a compound of formula XV,
L⁶-Y^{b} XV
wherein Y^{b} represents —C(O)OR^{9a}, in which R^{9a} is other than H, and L⁶ represents a suitable leaving group;
(x) for compounds of formula I in which Y¹ and/or, if present, Y^{1a} represent —C(O)OR^{9a} in which R^{9a} is H, reaction of a compound of formula XIV as defined above but in which L⁵ and/or L^{5a} (as appropriate) represents either:
(I) an alkali metal (for example, such as one defined in respect of process step (ix) above); or
(II) —Mg-halide,
with carbon dioxide, followed by acidification;
(xi) for compounds of formula I in which Y¹ and/or, if present, Y^{1a} represent —C(O)OR^{9a}, reaction of a corresponding compound of formula XIV as defined above but in which L⁵ and/or L^{5a} (as appropriate) is a suitable leaving group with CO (or a reagent that is a suitable source of CO), in the presence of a compound of formula XVI,
R^{9a}OH XVI
wherein R^{9a} is as defined above;
(xia) for compounds of formula I in which Y¹ and/or, if present, Y^{1a} represent —C(O)OH, reaction of a compound of formula XVIA, 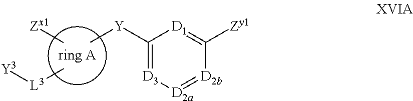
wherein at least one of Z^{x1} and Z^{y1} represents —CN, and the other may also represent —CN, or —Y¹ or —Y^{1a} (or hydrogen; as appropriate), and ring A, Y¹, Y^{1a}, D₁, D₂ₐ, D_{2b}, D₃, Y, L³ and Y³ are as defined above;
(xii) for compounds of formula I in which Y represents —C≡C—, reaction of either a compound of formula XVII or XVIII, 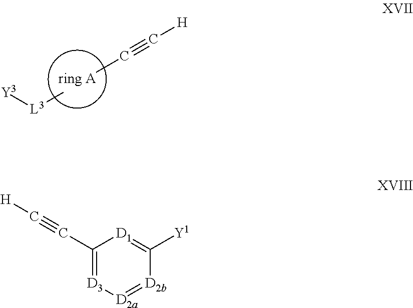
respectively with a compound of formula XIX or XX, 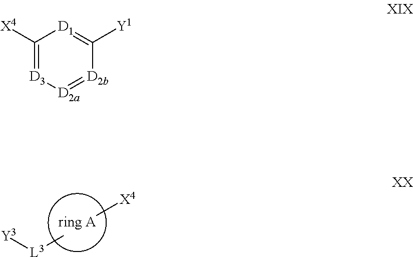
or protected derivatives thereof, wherein (in all cases) X⁴ represents a suitable leaving group such as one defined above in respect of Z^{x} and Z^{y}, and ring A, D₁, D₂ₐ, D_{2b}, D₃, Y¹, L³ and Y³ are as defined in claim 1;
(xiii) for compounds of formula I in which Y represents —CH═CH—, either:
(a) reaction of either a compound of formula XXI or XXII, 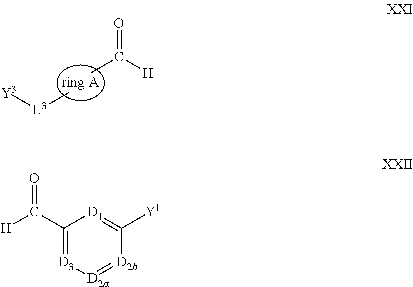
respectively with a compound of formula XXIII or XXIV, 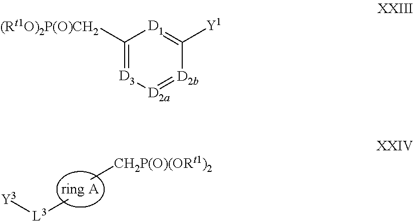
or protected derivatives thereof (e.g. amino-protected derivatives) wherein (in all cases) R^{t1} represents, at each occurrence when used herein, C₁₋₆ alkyl optionally substituted by one or more halo atoms, and ring A, D₁, D₂ₐ, D_{2b}, D₃, Y¹, L³ and Y³ are as defined in claim 1;
(b) reaction of either a compound of formula XXI or XXII as defined above, respectively with a compound of formula XXV or XXVI, 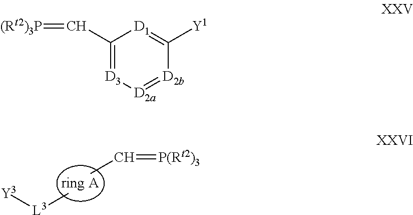
or protected derivatives thereof, wherein R^{t2} represents, at each occurrence when used herein, aryl (optionally substituted as defined by the integer B in claim 1) or C₁₋₆ alkyl optionally substituted by one or more halo atoms, and ring A, D₁, D₂ₐ, D_{2b}, D₃, Y¹, L³ and Y³ are as defined in claim 1;
(c) reduction of a corresponding compound of formula I in which Y represents —C≡C—;
(d) reaction of either a compound of formula XXVIA or XXVIB, 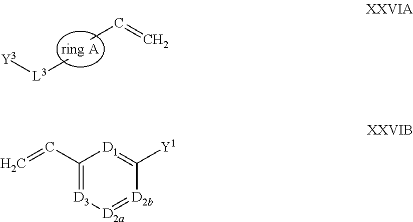
respectively with a compound of formula XXVIC or XXVID, 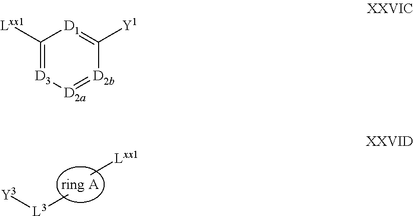
or protected derivatives thereof wherein (in all cases) L^{xx1} represents a suitable leaving group, and ring A, D₁, D₂ₐ, D_{2b}, D₃, Y¹, L³ and Y³ are as defined in claim 1;
(xiv) for compounds of formula I in which Y represents —CH₂—CH₂—, reduction of a corresponding compound of formula I in which Y represents —C≡C— or —CH═CH—;
(xv) for compounds of formula I in which Y represents —N(R^{f1})C(O)—, —C(O)—N(R^{g1})—, —N(Rⁿ¹)—S(O)₂— or —S(O)₂—N(R^{p1}), reaction of either a compound of formula XXVII or XXVIII, 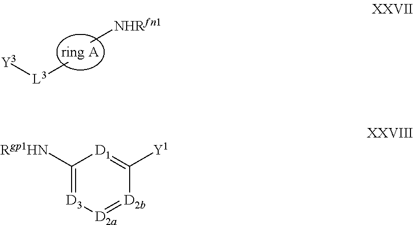
wherein R^{fn1} represents R^{f1} or Rⁿ¹ (as appropriate) and R^{gp1} represents R^{g1} or R^{p1} (as appropriate), respectively with a compound of formula XXIX or XXX, 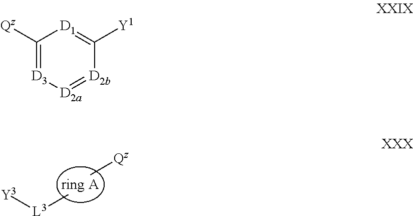
or protected derivatives thereof, wherein Q^{z} represents, —C(O)L^{xx} or —S(O)₂L^{x1} (in which L^{xx} and L^{x1} independently represent —OH or a leaving group), as appropriate, and ring A, D₁, D₂ₐ, D_{2b}, D₃, R^{f1}, R^{g1}, Rⁿ¹, R^{p1}, Y¹, L³ and Y³ are as defined in claim 1;
(xvi) for compounds of formula I in which Y represents —C(R^{k1})(R^{k2})—N(R^{m1})— or —N(Rⁱ¹)—C(R^{j1})(R^{j2})— either:
(a) for those compounds in which R^{k1}, R^{k2}, R^{m1}, Rⁱ¹, R^{j1} and R^{j2} represent hydrogen, reaction of a compound of formula XXVII or XXVIII in which R^{fn1} and R^{gn1} both represent hydrogen, respectively with a compound of formula XXXI or XXXII, 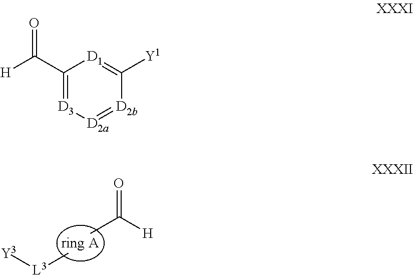
or protected derivatives thereof, wherein ring A, D₁, D₂ₐ, D_{2b}, D₃, Y¹, L³ and Y³ are as defined in claim 1;
(b) reaction of a compound of formula XXVII or XXVIII, respectively with a compound of formula XXXIA or XXXIIA, 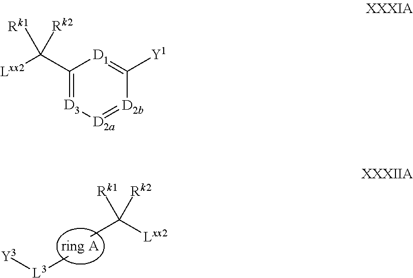
or protected derivatives thereof, wherein L^{xx2} represents a suitable leaving group such as one hereinbefore defined in respect of L^{xx1}, ring A, D₁, D₂ₐ, D_{2b}, D₃, Y¹, L³, Y³, R^{k1} and R^{k2} are as defined in claim 1;
(c) reaction of either a compound of formula XXXIII or XXXIV, 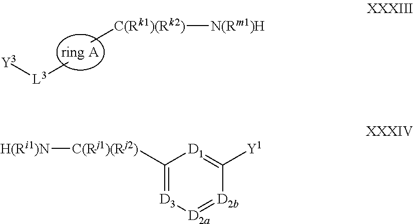
respectively with a compound of formula XIX or XX, as defined above, or protected derivatives thereof, wherein ring A, D₁, D₂ₐ, D_{2b}, D₃, Y¹, R^{j1}, R^{j2}, R^{k1}, R^{k2}, Rⁱ¹, R^{m1}, L³ and Y³ are as defined in claim 1;
(xvii) for compounds of formula I in which Y represents —O—C(R^{d1})(R^{d2})— or —C(R^{e1})(R^{e2})—O—, reaction of either a compound of formula XXXV or XXXVI, 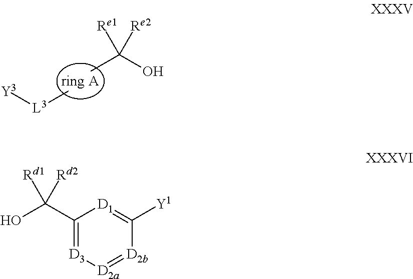
respectively with a compound of formula XIX or XX, as defined above, or protected derivatives thereof, wherein ring A, D₁, D₂ₐ, D_{2b}, D₃, Y¹, R^{d1}, R^{d2}, R^{e1}, R^{e2}, L³ and Y³ are as defined in claim 1;
(xviii) for compounds of formula I in which Y represents —CH(OH)—, reduction of a compound of formula XXXVII, 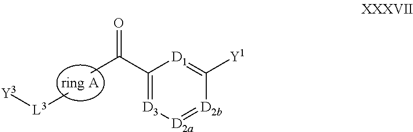
or protected derivatives thereof, wherein ring A, D₁, D₂ₐ, D_{2b}, D₃, Y¹, L³ and Y³ are as defined in claim 1;
(xix) for compounds of formula I in which Y represents a direct bond, reaction of a compound of formula XXXVIII or XXXIX, 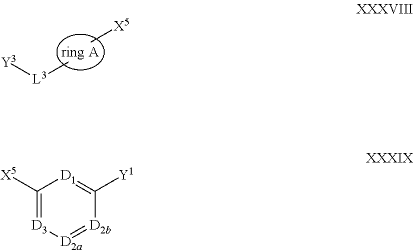
respectively with a compound of formula XIX or XX, as defined above, wherein X⁵ represents a group defined above in respect of Z^{x} and Z^{y}, a metal halide, or a magnesium halide, a stannane, an organoboronic acid, or an organosilane, and ring A, D₁, D₂ₐ, D_{2b}, D₃, Y¹, L³ and Y³ are as defined in claim 1;
(xx) for compounds of formula I in which R^{9a} represents hydrogen, formylation of a compound of formula XL, 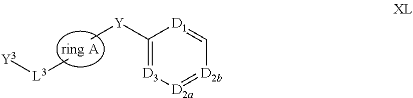
followed by oxidation under standard conditions;
(xxi) for compounds of formula I in which L² or L³ represent —N(H)—CH₂—, reductive amination of a compound of formula II as defined above, with a compound of formula XLI,
Y^{a}—C(O)H XLI
wherein Y^{a} is as defined in claim 1;
(xxii) reaction of a compound of formula XL, as defined above, with phosgene or triphosgene in the presence of a Lewis acid, followed by reaction in the presence of a compound of formula XVI as defined above;
(xxiii) for compounds of formula I in which L² and/or L³ represent —OA¹⁷-, reaction of a compound of the formula XLII, 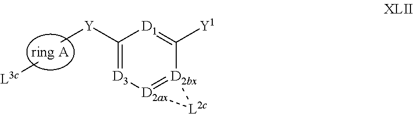
or a protected derivative thereof, wherein one of D₂ₐₓ and D_{2bx} represents D₂ and the other represents —C(-L^{2c})=, L^{2c} represents —OH or -L²-Y², L^{3c} represents -L³-Y³, provided that at least one of L^{2c} and L^{3c} represents —OH, and Y, ring A, D₁, D₂, D₃ and Y¹ are as defined in claim 1, with:
(a) when A¹⁷ represents a direct bond, a compound of formula XLIII,
Y^{c}—X^{c} XLIII
wherein Y^{c} represents Y² or Y³ as defined in claim 1, and X^{c} represents a suitable leaving group;
(b) when A¹⁷ represents —CH₂—, with a compound of formula XLIV,
Y^{c}—CH₂—X^{d} XLIV
wherein X^{d} represents a suitable leaving group and Y^{c} is as defined above;
(xxiv) for compounds of formula I in which Y represents —C(OR^{q1})(R^{h1})— and R^{q1} is other than hydrogen, reaction of a corresponding compound of formula I in which R^{q1} is hydrogen, with either:
(a) a compound of formula XLIVA,
R^{q1a}—OH XLIVA
wherein R^{q1a} represents R^{q1} provided that it does not represent hydrogen;
(b) an appropriate acylating reagent (when there is a ═O substituent α to the oxygen atom) or an appropriate sulfonylating reagent (when R^{q1a} is —S(O)₂R^{r1}).

32. A process for the preparation of a pharmaceutical formulation as defined in claim 21, which process comprises bringing into association a compound of formula I, as defined in claim 1 but without all of the provisos except (A)(I)(a), (A)(I)(d) and (B)(II), or a pharmaceutically acceptable salt thereof with a pharmaceutically-acceptable adjuvant, diluent or carrier.

33. A process for the preparation of a combination product as defined in any one of claims 28 to 30, which process comprises bringing into association a compound of formula I, as defined in claim 1 but without the provisos, or a pharmaceutically acceptable salt thereof with the other therapeutic agent that is useful in the treatment of a respiratory disorder and/or inflammation, and at least one pharmaceutically-acceptable adjuvant, diluent or carrier.
